(19) United States
(12) Patent Application Publication
(10) Pub. No.: US 2008/0095699 A1
(43) Pub. Date: Apr. 24, 2008

(54) IMAGING CONTRAST AGENTS USING NANOPARTICLES

(76) Inventors: Shiying Zheng, Webster, NY (US); Ruizheng Wang, Rochester, NY (US); Wenyi Che, Rochester, NY (US)

Correspondence Address: Patent Legal Staff;Eastman Kodak Company, 343 State Street, Rochester, NY, 14650-2201, US

(21) Appl. No.: 11/738,558

(22) Filed: Apr. 23, 2007

Related U.S. Application Data
(60) Provisional application No. 60/862,229, filed on Oct. 20, 2006.

(51) Int. Cl. A61K 49/00; A61K 39/395; A61K 49/06; A61K 49/18; A61K 51/12; A61K 9/14

(52) U.S. Cl. 424/1.11; 424/141.1; 424/489; 424/9.1; 424/9.3; 424/9.32

(57) ABSTRACT

The present invention relates to a nanoparticle comprising self-assembled crosslinked, amphiphilic block copolymers and at least one immobilized dye, wherein the self-assembled, crosslinked, amphiphilic block copolymers comprise a hydrophilic block and a hydrophobic block, wherein the self-assembled, crosslinked, amphiphilic block copolymers are self-assembled to form a core of the nanoparticle comprising a hydrophobic block, wherein the hydrophobic block is derived from at least one pendant multifunctional crosslinked alkoxy silane or amino silane moiety, and an exterior of the nanoparticle comprising a hydrophilic block, and wherein the immobilized dye is immobilized in the core.

### FIELD OF THE INVENTION

The present invention relates to nanoparticles derived from self-assembly of amphiphilic copolymers to form crosslinked particles with dye immobilized in the particle.

### BACKGROUND OF THE INVENTION

The ordered assembly of nanoscale and molecular components has promise to create molecular-assemblies capable of mimicking biological function, and capable of interacting with living cells and cellular components. Many techniques for creating nanoscale assemblies are being developed and include small-molecule assembly, polyelectrolyte assembly, nanoscale precipitation, core-shell assemblies, heterogeneous precipitation, and many others. One of the great challenges in materials science is the creation of defined structure and tuning the function in molecular level and the integration of nanoscale assemblies into living organisms. Successful integration requires assemblies which are colloidally stable under highly specific conditions (physiological pH and ionic strength), are compatible with blood components, are capable of avoiding detection by the immune system, and survive the multiple filtration and waste removal systems inherent to living organisms. Highly precise methods of assembly are necessary for building ordered nanoscale assemblies capable of performing under stringent conditions.

Self-assembly is ubiquitous in nature and has provided an insight to construct artificial nanosized structure. The formation of core-shell polymeric nanoparticles through the association of amphiphilic polymers has been an intense field of research over the last few decades. It is well known that, in the presence of a solvent or solvent mixture that is selective for one block, amphiphilic block copolymers have the ability to assemble into colloidal aggregates of various morphologies. In particular, significant interest has been focused on the formation of polymeric micelles and nanoparticles from amphiphilic block or graft copolymers in aqueous media. This organized association occurs as polymer chains reorganize to minimize interactions between the insoluble hydrophobic blocks and water. The resulting nanoparticles possess cores composed of hydrophobic block segments surrounded by outer shells of hydrophilic block segments. The core-shell structures of amphiphilic micellar assemblies have been utilized as novel carrier systems in the field of drug delivery.

More recently, there has been intense interest focused upon developing surface-modified nanoparticulate materials that are capable of carrying biological, pharmaceutical or diagnostic components. The components, which might include drugs, therapeutics, diagnostics, and targeting moieties can then be delivered directly to diseased tissue or bones and be released in close proximity to the disease and reduce the risk of side effects to the patient. This approach has promised to significantly improve the treatment of cancers and other life threatening diseases and may revolutionize their clinical diagnosis and treatment. The components that may be carried by the nanoparticles can be attached to the nanoparticle by well-known bio-conjugation techniques; discussed at length in Bioconjugate Techniques, G. T. Hermanson, Academic Press, San Diego, Calif. (1996). The most common bio-conjugation technique involves conjugation, or linking, to an amine functionality.

Certain nanoparticles were recently proposed as carriers for certain pharmaceutical agents. See, e.g., Sharma et al. Oncology Research 8, 281 (1996); Zobel et al. Antisense Nucl. Acid Drug Dev., 7:483 (1997); de Verdiere et al. Br. J. Cancer 76, 198 (1997); Hussein et al., Pharm. Res., 14, 613 (1997); Alyautdin et al. Pharm. Res. 14, 325 (1997); Hrkach et al., Biomaterials, 18, 27 (1997); Torchilin, J. Microencapsulation 15, 1 (1988); and literature cited therein. The nanoparticle chemistries provide for a wide spectrum of rigid polymer structures, which are suitable for the encapsulation of drugs, drug delivery and controlled release. Some major problems of these carriers include aggregation, colloidal instability under physiological conditions, low loading capacity, restricted control of the drug release kinetics, and synthetic preparations which are tedious and afford very low yields of product.

Many authors have described the difficulty of making colloidally stable dispersions of colloids having surface modified particles, achieving colloidal stability under physiological conditions (pH 7.4 and 137 mM NaCl) is yet even more difficult. Burke and Barret (Langmuir, 19, 3297(2003)) describe the adsorption of the amine-containing polyelectrolyte, polyallylamine hydrochloride, onto 70-100 nm silica particles in the presence of salt. The authors state (p.3299) “the concentration of NaCl in the colloidal solutions was maintained at 1.0 mM because higher salt concentrations lead to flocculation of the colloidal suspension”.

Siiman et al. U.S. Pat. No. 5,248,772 describes the preparation of colloidal metal particles having a cross-linked aminodextran coating with pendant amine groups attached thereto. The colloid is prepared at a very low concentration of solids 0.24% by weight, there is no indication of the final particle size, and there is no indication of the fraction of aminodextran directly bound to the surface of the colloid. Since the ratio of the weight of shell material (0.463 g) to the weight of core material (0.021 g) in example 2 is roughly 21:1, it appears likely that only a very small fraction of the aminodextran is bound to the surface of the colloid and that most remains free in solution. There is a problem in that this leads to a very small amount of active amine groups on the surface of the particle, and hence a very low useful biological, pharmaceutical or diagnostic components capacity for the described carrier particles in the colloids. There is an additional problem in that polymer not adsorbed to the particle surfaces may interfere with subsequent attachment or conjugation, of biological, pharmaceutical or diagnostic components. This reference, however, describes solid metal particles with a biocompatibilizing coating, which is fundamentally different from the nanoparticles derived from amphiphilic copolymer micelles of this invention.

U.S. Pat. No. 6,207,134 B1 describes particulate diagnostic contrast agents comprising magnetic or supermagnetic metal oxides and a polyionic coating agent. The coating agent can include “physiologically tolerable polymers” including amine-containing polymers. The contrast agents are said to have “improved stability and toxicity compared to the conventional particles” (col. 6, line 11-13). The authors state (Col. 4, line 15-16) that “not all the coating agent is deposited, it may be necessary to use 1.5-7, generally about two-fold excess . . . ” of the coating agent. The authors further show that only a small fraction of polymer adsorbs to the particles. For example, from FIG. 1 of '134, at 0.5 mg/mL polymer added only about 0.15 mg/mL adsorbs, or about 30%. The surface-modified particles of '134 are made by a conventional method involving simple mixing, sonication, centrifugation and filtration. Again, this describes polymer-coated solid metal particles, which are fundamentally different from the nanoparticles derived from amphiphilic copolymer micelles described herein.

U.S. Pat. No. 5,078,994 discloses a copolymer microparticle, prepared by emulsion polymerization, which is derived from at least about 5 weight percent of free carboxylic acid group-containing vinyl monomers, monomers which have a poly(alkylene oxide) appended thereto, oleophilic monomers and other nonionic hydrophilic monomers. Microgels containing these copolymers having a median water swollen diameter of about 0.01 to about 1.0 micrometer are disclosed. Pharmaceutical and diagnostic compositions are disclosed comprising a therapeutic or diagnostic agent and microgels comprising a copolymer derived from at least about 5 weight percent of non-esterified carboxylic acid group-containing vinyl monomers, oleophilic monomers and other nonionic hydrophilic monomers, with the proviso that when the median water swollen diameter of the microgels is 0.1 micrometer or greater, at least 5 weight percent of the monomers have a poly(alkylene oxide) appended thereto. Diagnostic and therapeutic methods are also disclosed wherein the microgels are substantially protein non-adsorbent and substantially refractory to phagocytosis. These particles, however, contain a large fraction of hydrophobic monomers and a low degree of PEGylation, and thus have inferior colloidal stability and biocompatibility.

US 2003/0211158 discloses novel microgels, microparticles, typically 0.1-10 microns in size, and related polymeric materials capable of delivering bioactive materials to cells for use as vaccines or therapeutic agents. The materials are made using a crosslinker molecule that contains a linkage cleavable under mild acidic conditions. The crosslinker molecule is exemplified by a bisacryloyl acetal crosslinker. The new materials have the common characteristic of being able to degrade by acid hydrolysis under conditions commonly found within the endosomal or lysosomal compartments of cells thereby releasing their payload within the cell. The materials can also be used for the delivery of therapeutics to the acidic regions of tumors and sites of inflammation. These particles, however, are of a large enough size range that uptake by the reticuloendothelial system can be expected to be a problem. In addition, the degree of PEGylation is low and in-vivo agglomeration has been identified as a problem (see Kwon, Y. J.; Standley, S. M.; Goh, S. L.; Frechet, J. M. J. Journal of Controlled Release 2005, 105, 199-212.)

U.S. Pat. No. 6,333,051 discloses copolymer networks having at least one cross-linked polyamine polymer fragment and at least one nonionic water-soluble polymer fragment, and compositions thereof, having at least one suitable biological agent. The invention relates to polymer technology, specifically polymer networks having at least one cross-linked polyamine polymer fragment and at least one nonionic water-soluble polymer fragment, and compositions thereof. These nanogels, however, differ from those of this invention in that they are not based on an ethylenically unsaturated backbone. In addition, the preparation of these nanogels is tedious and affords only small quantities.

The Journal of the American Chemical Society 124(51): 15198-15207 (“Polymeric Nanogels Produced via Inverse Microemulsion Polymerization as potential Gene and Antisense Delivery Agents”) describes crosslinked acrylate nanogels with quaternary amine functionalities and PEGDA crosslinker. The nanogels are approximately 40-200 nm in size. These nanogels, however, do not contain sufficient PEGylation and the preparation is tedious and only affords small quantities.

U.S. Pat. No. 5,874,111 discloses the preparation of highly monodispersed polymeric hydrophilic nanogels having a size of up to 100 nm, which may have drug substances encapsulated therein. The process comprises subjecting a mixture of an aqueous solution of a monomer or preformed polymer reverse micelles, a cross linking agent, initiator, and optionally, a drug or target substance to polymerization. The polymerized reaction product is dried for removal of solvent to obtain dried nanoparticles and surfactant employed in the process of preparing reverse micelles. The dry mass is dispersed in aqueous buffer and the surfactant and other toxic material are removed therefrom. This invention relates to a process for the preparation of highly monodispersed polymeric hydrophilic nanoparticles with or without target molecules encapsulated therein and having sizes of up to 100 nm and a high monodispersity. Again, these particles do not contain sufficient PEGylation to afford biocompatibility and the preparation is tedious.

Kataoka et al. (Advanced Drug Delivery Reviews 47, 113, 2001) described the use of block copolymer micelles for imaging and drug delivery. Block copolymers with amphiphilic character, having a large solubility difference between hydrophilic and hydrophobic segments, are known to assemble in an aqueous milieu into polymeric micelles with a microscopic size range. These micelles have a fairly narrow size distribution and are characterised by their unique core-shell architecture, where hydrophobic segments are segregated from the aqueous exterior to form an inner core surrounded by a palisade of hydrophilic segments. Recently, interest has been raised in the application of these block copolymer micelles as novel carrier systems in the field of drug targeting because of the high drug-loading capacity of the inner core as well as of the unique disposition characteristics in the body. The micelles of Kataoka can dissociate under high dilution. Kataoka does not disclose micelles that are core-crosslinked with the dye immobilized in the core and that do not dissociate under dilution. When the dye is immobilized, fluorescence quantum yield is also enhanced.

U.S. Pat. No. 5,429,826 discloses chemically fixing the core of the micelle to improve the stability of the micellar aggregates in aqueous environment. The disclosed copolymers contain crosslinkable end-groups for crosslinking. Because the end group useful for crosslinking comes from endcapping the chain end of the polymerization, the conversion yield can be low and each polymer chain has only maximum one end-group useful for crosslinking. The crosslinking efficiency is rather limited and requires long reaction time, for example, 5 days. The patent also discloses copolymers containing pendant multifunctional vinyl groups for crosslinking. The crosslinking requires a free radical initiator with either UV radiation or high reaction temperature for prolonged reaction time to induce crosslinking. Most biological agents and dyes incorporated in the micelles will decompose under such conditions. The near infrared dyes are especially sensitive to photooxidation and photostability is generally poor (Li, J.; Chen, P.; Hu, X. J.; Zheng, D. S.; Okasaki, T.; Hayami, M. Chinese Chem. Lett. 1996, 7 (12), 1121-1124. The patent does not disclose the incorporation of functional groups into the shell of the micelles for forming a linkage between a biological, pharmaceutical or diagnostic component of interest and a nanoparticle.

Kataoka et al. (Advanced Drug Delivery Reviews 47, 113, 2001) also discloses the stabilization of the polymeric micelle by cross-linking of the core or the shell of the micelle to suppress the dissociation of the micelle. (See A. Guo, G. Liu, J. Tao, Star polymers and nanospheres from cross-linkable diblock copolymers, Macromolecules 29 (1996) 2487-2493; see also K. B. Thurmond, T. Kowalewski, K. L. Wooley, Water-soluble needle-like structures: the preparation of shell-cross-linked small particles, J. Am. Chem. Soc. 118 (1996) 7239-7240.) Cross-linking by reversible bonds is also described, wherein the bond is cleaved in response to physical or chemical stimuli at the site of drug action. Micelles with cores composed of PEG-PLys and oligo-DNA cross-linked by disulfide bonds have been observed to cleave within the cell because the intracellular compartment has a stronger reducing environment than the extracellular fluid, resulting in micelles with a tailored property to promptly dissociate under the physiological salt conditions found inside cells. (Y. Kakizawa, A. Harada, K. Kataoka, Environment-sensitive stabilization of core-shell structured polyion complex micelle by reversible cross-linking of the core through disulfide bond, J. Am. Chem. Soc. 121 (1999) 11247-11248.).

The present invention describes the preparation and application of nanoparticles derived from core-crosslinked micelles, in which the hydrophobic core is derived from pendant multifunctional crosslinked alkoxy silane or amino silane moiety to form very stable, inorganic silicon oxide rich material and the shell is derived from hydrophilic segments, and is similar to an organic/inorganic hybrid material.

The conventional sol-gel process occurs in liquid solution of organometallic precursors (tetramethoxysilane, tetraethoxysilane, Zr(IV)-propoxide, Ti(IV)-butoxide, etc.), which, by means of hydrolysis and condensation reactions, lead to the formation of a new phase (SOL).

M-O—R+H₂O [symbol] M-OH+R—OH (hydrolysis) (M=Si, Zr, Ti)

M-OH+HO-M→M-O-M+H₂O (water condensation)

M-O—R+HO-M→M-O-M+R—OH (alcohol condensation)

The SOL is made of solid particles of a very small diameter suspended in a liquid phase. The particles then condense in a new phase (GEL) in which a solid macromolecule is immersed in a liquid phase (solvent).

The fundamental property of the sol-gel process is that it is possible to generate ceramic material at a temperature close to room temperature. Therefore such a procedure opened the possibility of incorporating in these glasses soft dopants, such as fluorescent dye molecules and organic chromophores. The Sol-Gel process allows the synthesis of ceramic materials of high purity and homogeneity by means of preparation techniques different from the traditional process of fusion of oxides.

The present invention takes advantage of the sol-gel process to induce crosslinking in the core of the micellar nanoparticles to form stable network of silicon oxide. In particular, this process allows for incorporation of fluorescent dye molecules in the core of the nanoparticles by covalent chemical bonding.

### PROBLEM TO BE SOLVED

It would be desirable to produce nanoparticles for use as carriers for bioconjugation and targeted delivery, which are stable so that they can be injected in vivo, especially intravascularly. Further, it is desirable that the nanoparticles for use as carriers be stable under physiological conditions (pH 7.4 and 137 mM NaCl). Still further, it is desirable that the particles avoid detection by the immune system. In addition, it would be desirable to produce nanoparticles as novel imaging contrast agent for Optical Molecular Imaging which demonstrate well-controlled particle size and brightness, resist protein adsorption, have convenient attachment moieties for the attachment of biological targeting units, and contain emissive dyes that emit in the infrared (IR).

### SUMMARY OF THE INVENTION

The present invention relates to a nanoparticle comprising self-assembled crosslinked, amphiphilic block copolymers and at least one immobilized dye, wherein the self-assembled, crosslinked, amphiphilic block copolymers comprise a hydrophilic block and a hydrophobic block, wherein the self-assembled, crosslinked, amphiphilic block copolymers are self-assembled to form a core of the nanoparticle comprising the hydrophobic block, wherein the hydrophobic block is derived from at least one pendant multifunctional crosslinked alkoxy silane or amino silane moiety, and an exterior of the nanoparticle comprising the hydrophilic block, and wherein the immobilized dye is immobilized in the core.

### ADVANTAGEOUS EFFECT OF THE INVENTION

The present invention includes several advantages, not all of which are incorporated in a single embodiment. The materials of the present invention provide a medium for high loading levels of dyes, are stable within a broad window of conditions, are easy to prepare, and demonstrate high biological compatibility. In addition, the process used to form the present particles utilizes mild conditions, allowing the use of dyes and other compounds to be carried by the particles, which would otherwise be damaged or destroyed by the processing conditions.

### BRIEF DESCRIPTION OF THE DRAWINGS

FIG. 1 illustrates Protein binding assay of nanoparticles, run on 10% SDS- polyacrylamide gel electrophoresis (SDS-PAGE) gel.

FIG. 2 illustrates a cytotoxicity assay of nanoparticles to Human umbilical vein endothelial cells (HUVEC) cells.

### DETAILED DESCRIPTION OF THE INVENTION

The present invention relates to a nanoparticle comprising self-assembled crosslinked, amphiphilic block copolymers and at least one immobilized dye, wherein the self-assembled, crosslinked, amphiphilic block copolymers comprise a hydrophilic block and a hydrophobic block, wherein the self-assembled, crosslinked, amphiphilic block copolymers are self-assembled to form a core of the nanoparticle comprising the hydrophobic block and an exterior of the nanoparticle comprising the hydrophilic block, and wherein the immobilized dye is immobilized in the core.

The present invention describes the preparation and application of nanoparticles derived from core-crosslinked micelles. These nanoparticles have improved stability. The hydrophobic core is derived from pendant multifunctional crosslinkable alkoxy silane or amino silane moieties to form very stable, inorganic silicon oxide rich material and the shell is derived from hydrophilic segments, and it is similar to an organic/inorganic hybrid material. The dyes and/or other useful agents are immobilized in the core via covalent chemical bonding. The crosslinking can be accomplished under mild condition to prevent the decomposition of the dye or other useful agents. Most specifically, the crosslinking is accomplished at lower levels of heat and light and with less crosslinker, preserving dyes and other carried materials, which would be destroyed by contact with conventional heat and light levels previously used in preparing similar particles.

The present invention describes the preparation and application of nanoparticles derived from core-crosslinked micelles, in which the hydrophobic core is derived from pendant multifunctional crosslinkable alkoxy silane or amino silane moiety through a sol-gel process to form very stable, inorganic silicon oxide rich material and the shell is derived from hydrophilic segments, and is similar to an organic/inorganic hybrid material.

The present invention takes advantage of the sol-gel process to induce crosslinking in the core of the micellar nanoparticles to form stable network of silicon oxide. In particular, this process allows for incorporation of fluorescent dye molecules in the core of the nanoparticles by covalent chemical bonding.

Organic/inorganic nanoparticles have attracted considerable interest due to the fascinating size-dependent optical, magnetic, and electronic properties of particles at the nanoscale. Hybrid nanoparticles consist of a polymer with incorporated inorganic nanoparticles; the organic polymer shell determines the chemical properties of nanoparticles, the interaction with the environments, and their responsiveness to external stimuli, while their physical properties are governed by both the size and shape of the inorganic core and surrounding organic layer.

Whenever used in the specification the terms set forth shall have the following meaning:

The term nanoparticle or nanoparticulate refers to a particle with a size of less than 100 nm.

The term “biocompatible” means that a composition does not disrupt the normal function of the bio-system into which it is introduced. Typically, a biocompatible composition will be compatible with blood and does not otherwise cause an adverse reaction in the body. For example, to be biocompatible, the material should not be toxic, immunogenic or thrombogenic.

The term “biodegradable” means that the material can be degraded either enzymatically or hydrolytically under physiological conditions to smaller molecules that can be eliminated from the body through normal processes.

The “stable dispersion” means that the solid particulates do not aggregate, as determined by particle size measurement, and settle from the dispersion, usually for a period of hours, preferably weeks to months. Terms describing instability include aggregation, agglomeration, flocculation, gelation and settling. Significant growth of mean particle size to diameters greater than about three times the core diameter, and visible settling of the dispersion within one day of its preparation is indicative of an unstable dispersion.

### Nanoparticle

The nanoparticle is a self-assembled micelle formed from amphiphilic block copolymers.

Self-assembly is the fundamental principle which generates structural organization on all scales from molecules to galaxies. It is defined as reversible processes in which pre-existing parts or disordered components of a preexisting system form structures of patterns. Self-assembly can be classified as either static or dynamic. Static self-assembly is when the ordered state occurs when the system is in equilibrium and does not dissipate energy. Dynamic self-assembly is when the ordered state requires dissipation of energy. There are many examples of self-assembling system and the most well-studied subfield of self-assembly is molecular self-assembly.

Molecular self-assembly is the assembly of molecules without guidance or management from an outside source. There are two types of self-assembly, intramolecular self-assembly and intermolecular self-assembly. Intramolecular self-assembling molecules are often complex polymers with the ability to assemble from the random coil conformation into a well-defined stable structure (secondary and tertiary structure). An example of intramolecular self-assembly is protein folding. Intermolecular self-assembly is the ability of molecules to form supramolecular assemblies (quarternary structure). A simple example is the formation of a micelle by surfactant molecules in solution.

Self-assembly can occur spontaneously in nature, for example in cells (such as the self-assembly of the lipid bilayer membrane) and other biological systems, as well as in human engineered systems. It usually results in the increase in internal organization of the system. Biological self-assembling systems, including synthetically engineered self-assembling peptides and other biomaterials, have been shown to have superior handling, biocompatibility and functionality. These advantages are due directly to self-assembly from biocompatible precursors creating biomaterials engineered at the nano-scale. Thus, it is most desireable in material science to synthesize molecules for self-assembly chemical process.

Amphiphilic block or graft copolymers form micelles in selective solvents, which are thermodynamically good solvents for one block and poor solvents for the other. The free energy of the system is lowered in such solvents by micellar association compared to dispersed single chains. The micelles consist of a compact core of the insoluble block with a soluble corona consisting of the second block. Compared to surfactant micelles, polymeric micelles are generally more stable, with a remarkably lowered critical micellar concentration (CMC), and have a slower rate of dissociation, allowing retention of loaded drugs in drug delivery for a longer period of time and eventually achieving higher accumulation of a drug at the target site.

The amphiphilic copolymers can be either block or graft copolymers. The molecular weights of the hydrophilic and hydrophobic components are not critical. A useful range of the molecular weight of the hydrophilic component is between 1,000 to 100,000, and preferably 2,000 and 60,000. The molecular weight of the hydrophobic component is between 500 and 100,000, and preferably between 1,000 and 80,000, more preferably between 2,000 and 50,000. The mole fractions of the component monomers may be determined from the recipe from which the copolymers was prepared or by any other suitable analytical method for determining polymer composition (NMR, titrations, etc).

Preferably, at least one block of the copolymers is derived from α,β-ethylenically unsaturated monomers. More preferably, at least one block of the copolymers is derived from α,β-ethylenically unsaturated monomers such as styrenes, (meth)acrylates or (meth)acrylamides. Most preferably, at least one block of the copolymers is derived from (meth)acrylates or styrenes.

### Core

Core segregation from aqueous milieu is the direct driving force for micellization and proceeds through a combination of intermolecular forces, including hydrophobic interaction, electrostatic interaction metal complexation, and hydrogen bonding of constituent block copolymers.

The hydrophobic core is derived from pendant multifunctional crosslinkable alkoxy silane or amino silane moieties. The core is crosslinked so that the nanoparticle is not capable of dissociation when diluted in a medium.

Hydrophobic components useful in the present invention include but are not limited to vinyl polymers, polyesters, polyamides, polyethers, polycarbonates, polyimides, and polycarbamates. Useful monomers for the hydrophobic components include but are not limited to α,β-ethylenically unsaturated monomers such as styrenics, acrylamides, and (meth)acrylates, lactones, lactams, lactic acid, and amino acids. Preferably, the hydrophobic components derived from styrenics, (meth)acrylamides, and (meth)acrylates. More preferably, the hydrophobic components derived from styrenics and (meth)acrylates containing crosslinkable alkoxy silane or amino silane groups.

### Exterior

A variety of hydrophilic polymers with a flexible nature can be selected as exterior-forming segments, which assemble into dense palisades of tethered chains to achieve effective steric stabilization propensities. The outer block consists in many cases of a polar poly(ethylene oxide) (PEO) block, which will form the shell of the nanocarrier and protect the core through steric stabilization. It has also been demonstrated that poly(ethylene oxide) prevents the adsorption of proteins and hence forms a biocompatible polymeric nanocarrier shell.

Useful hydrophilic components of the block copolymer in the present invention include but are not limited to poly(alkylene oxides) such as poly(ethylene oxide), poly(2-ethyloxazolines), poly(saccharides), dextrans and vinyl polymers containing poly(ethylene oxide) poly(ethylene oxide) moiety. Preferably hydrophilic components are poly(ethylene oxide) and vinyl polymers containing poly(ethylene oxide) moiety, and more preferably poly(ethylene oxide) poly(meth(acrylates)) containing poly(ethylene oxide) moiety, polystyrenes containing poly(ethylene oxide) moiety, poly(meth(acrymides) containing poly(ethylene oxide) moiety.

The size of these block copolymer micelles is determined by thermodynamic parameters, although partial size-control is possible by variation of the block length. These block copolymer micelles are typically several tenths of nanometers in diameter with a relatively narrow size distribution, and are therefore similar in size to viruses, lipoproteins, and other naturally occurring transport systems. A major obstacle for such nanocarrier systems is the non-specific uptake by reticuloendothelial systems (RES).

The size and the surface properties of such block copolymer based nanocarriers require careful design to achieve long circulation times in blood and to reach target sites. The polarity and functionality of each block allows control over the spontaneously formed core-shell architecture. While terminal functionalities on the outer block (“the shell”) control the biocompatibility and might also incorporate possible targeting functionalities of these nanocarriers, the inner block can be used to complex or covalently couple active drug and dye molecules.

### Particle size

The particle size(s) of the nanoparticle may be characterized by a number of methods, or combination of methods, including light-scattering methods, sedimentation methods, such as analytical ultracentrifugation, hydrodynamic separation methods, such as field flow fractionation, size exclusion chromatography, and electron microscopy. The nanogels in the examples were characterized primarily using light-scattering methods. Light-scattering methods can be used to obtain information regarding volume median particle diameter, the particle size number and volume distribution of nanogels, standard deviation of the distribution(s) and the distribution width.

The nanoparticle may have a volume average hydrodynamic volume median diameter of between 10 and 1000, preferably 10 to 100 nm as determined by quasi-elastic light scattering in phosphate buffered saline (137 mM NaCl, 2.7 mM KCl, 10 mM Na₂HPO₄, 2 mM KH₂PO₄ at pH 7.4.). Hydrodynamic diameter refers to the diameter of the equivalent sphere of the polymer and its associated solvent as determined by quasi-elastic light scattering.

The nanoparticle may also have a weight average molecular weight of from 10,000 to 6,000,000, preferably, from 40,000 to 1,000,000 and most preferably from 50,000 to 800,000 as measured by static light scattering or by size exclusion chromatography.

The amphiphilic block or graft copolymers consisting of hydrophilic and hydrophobic segments can be prepared via various living polymerization techniques such as anionic polymerization, cationic polymerization, group transfer polymerization, ring opening polymerization, and ring opening metathesis polymerization (ROMP) and controlled/living free radical polymerization.

Controlled/living radical polymerization has been explored as a means of producing well-defined polymers. Atom transfer radical polymerization (ATRP) involves the use of a novel initiating systems. The initiation system is based on the reversible formation of growing radicals in a redox reaction between various transition metal compounds and an initiator, for example alkyl halides, aralkyl halides or haloaklyl esters. It is one of the best methods to accomplish this because it can be applied to the polymerization of a wide variety of monomers (Wang, J.-S.; Matyjaszewski, K. J. Am. Chem. Soc. 1995, 117, 5614-5615; Matyjaszewski, K. J.; Wang, J.-S. U.S. Pat. No. 5,763,548). ARTP has great synthetic power to control the molecular architecture of polymers and is an exceptionally robust method of producing block or graft copolymers. Various amphiphilic polymers have been synthesized via atom transfer radical polymerization, and their amphiphilic properties can be well controlled.

Atom transfer radical polymerization is a versatile technique which offers several advantages over other polymerization routes including control over molecular weight and molecular weight distribution, and the polymers can be end-functionalized or block copolymerization upon the addition of other monomers.

Atom transfer radical polymerization is one of the most successful methods to polymerize styrenes, (meth)acrylates and a variety of other monomers in a controlled fashion, yielding polymers with molecular weights predetermined by the ratio of the concentrations of consumed monomer to introduced initiator and with low polydispersities. Because of its radical nature, atom transfer radical polymerization is tolerant to many functionalities in monomers leading to polymers with functionalities along the chains. Moreover, the initiator used determines the end groups of the polymers. By using a functional initiator, functionalities such as vinyl, hydroxyl, epoxide, cyano and other groups have been incorporated at one chain end, while the other chain end remains an alkyl halide and can be converted into other desired functional groups later. Not only does this feature offer tailorability of the polymer with a variety of compositions and functionalities, but this feature may be important in biomedical applications to modify the polymer shell on the nanoparticles with biological moieties for specific cellular interactions. With atom transfer radical polymerization, functionality and architecture can be combined, resulting in multifunctional polymers of different compositions and shapes such as block copolymers, multi-armed stars or hyperbranched polymers.

Also, atom transfer radical polymerization is a particularly successful controlled radical/living poymerization (CRP) method and has attracted commercial interest because of its easy experimental setup, use of readily accessible and inexpensive catalysts (usually copper complexes formed with aliphatic amines or imines, or pyridines, many of which are commercially available), and simple initiators (often alkyl halides). Atom transfer radical polymerization is probably the most robust and efficient CRP and well-defined polymers with controlled topology, composition and functionality are readily prepared.

### Immobilized Dye

The dyes contain functional groups that can react with the crosslinkable groups of the hydrophobic component of the copolymer and are immobilized in the core of the nanoparticles by covalent bonding. More specifically the dyes contain alkoxy silane or amino silane groups. Since the imaging dyes are immobilized in the nanoparticles, the quantum efficiency is enhanced. Dyes such as cyanine dyes tend to form aggregates that do not fluoresce and fluorescence quantum yield decreases. Immobilization of the dye in the core of the nanoparticle can reduce the aggregation and thus improve quantum efficiency.

Examples of suitable dyes include the following:

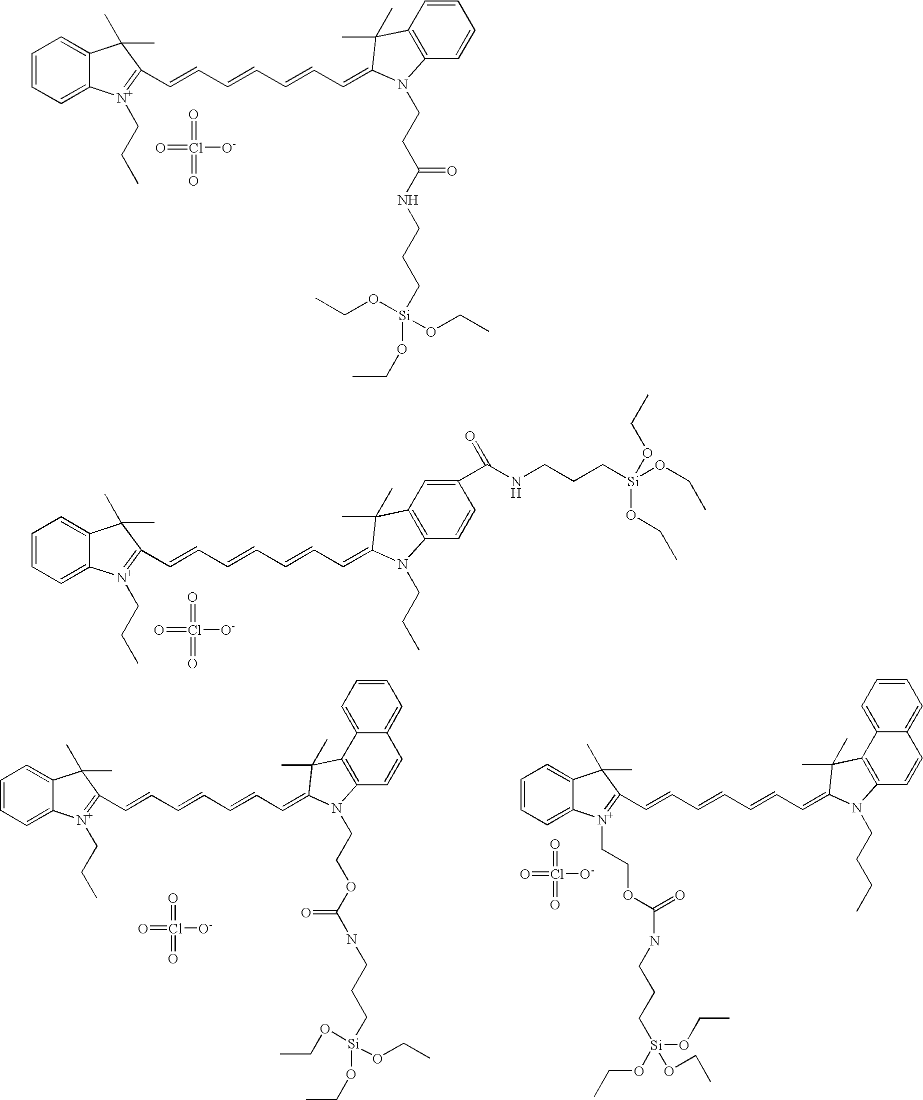

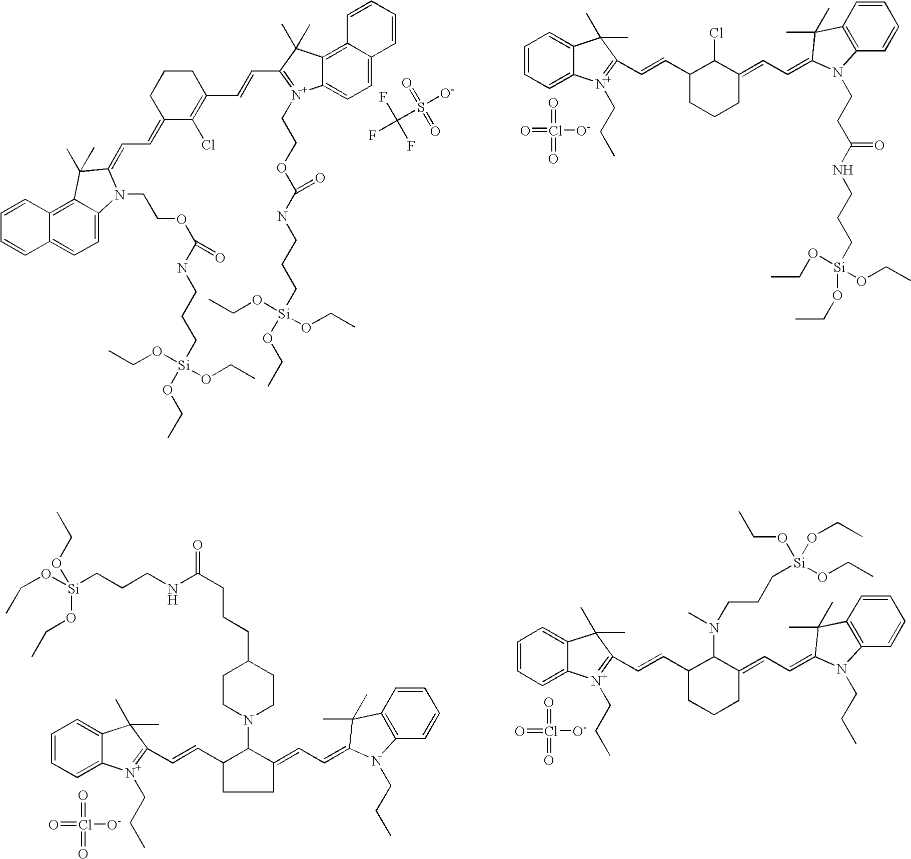

### Use as a Carrier, Biosensor or in Optical Molecular Imaging Applications

Dyes that are most useful as fluorescent biomarkers or contrast agents emit significant fluorescent light during in-vitro or in-vivo diagnostic procedures. Many dyes do not emit fluorescent light because excitation energy is emitted as heat or non-fluorescent light. Of those dyes that do emit fluorescent energy, many are self quenched due to aggregation effects or have low quantum yields. Suitable fluorescent dyes that accumulate in diseased tissue (above all, in tumors) and that show a specific absorption and emission behavior, may contribute towards enhancing the distinction of healthy from diseased tissue.

Examples of using dyes for in-vivo diagnostics in humans are photometric methods of tracing in the blood to determine distribution areas, blood flow, or metabolic and excretory functions, and to visualize transparent structures of the eye (ophthalmology). Preferred dyes for such applications are indocyanine green and fluorescein (Googe, J. M. et al., Intraoperative Fluorrescein Angiography; Ophthalmology, 100, (1993), 1167-70.

Indocyanine Green (Cardiogreen) is used for measuring the liver function, cardiac output and stroke volume, as well as the flood flow through organs and peripheral blood flows, (I. Med. 24 (1993), 10-27); in addition they are being tested as contrast media for tumor detection. Indocyanine green binds up to 100% to albumin and is mobilized in the liver. Fluorescent quantum efficiency is low in a hydrous environment. The LD50 (0.84 mmol/kg) is high enough that strong anaphylactic responses may occur. Indocyanine green is unstable when dissolved and cannot be applied in saline media because precipitation will occur.

Photosensitizers designed for used in photodynamic therapy (PDT) (including haematopoporphyrin derivatives, photophrin II, benzopopphyrins, tetraphenyl porphyrins, chlorines, phthalocyanines) were used up to now for localizing and visualizing tumors (Bonnett R.; New photosensitizers for the photodymanic therapy of tumors, SPIE Vol. 2078 (1994)). It is a common disadvantage of the compounds listed that their absorption in the wavelength range of 650-1200 nm is only moderate. The phototoxicity required for PDT is disturbing for purely diagnostic purposes. Other patent specifications dealing with these topics are U.S. Pat. No. 1,945,239, WO 84/04665, WO 90/10219, DE-OS 4136769, DE-PS 2910760.

Other dyes which have been developed for this purpose include: IRDye78, IrDye80, IRDye38, IRDye40, IRDye41, IRDye700, IRDye800, IRDye800CW, Cy5, Cy5.5, Cy7, IR-786, DRAQ5NO, Licor NIR, Alexa Fluor 680, Alexa Fluor 750, La Jolla Blue, quantum dots, as well as fluorphores described U.S. Pat. No. 6,083,875.

Typically, the dyes of the present invention are selected from the same family, such as the Oxonol, Pyryliuim, Squaric, Croconic, Rodizonic, polyazaindacenes or coumarins. Other suitable families of dyes include hydrocarbon and substituted hydrocarbon dyes; scintillation dyes (usually oxazoles and oxadiazoles); aryl- and heteroaryl-substituted polyolefins (C₂-C₈ olefin portion); merocyanines, carbocyanines; phthalocyanines; oxazines; carbostyryl; and porphyrin dyes. It is also possible, however, to achieve efficient energy transfer between different classes of dyes (dyes that are structurally different) such as between polyolefinic dyes and dipyrrometheneboron difluoride dyes, coumarin dyes and dipyrrometheneboron difluoride dyes, polyolefinic dyes and coumarin dyes; dipyrrometheneboron difluoride dyes and oxazine dyes; and many others.

Examples of commercially available dyes are listed below. Useful dyes of the present invention can be obtained from these dyes by further reaction to incorporate silane moieties for crosslinking. Useful parent dyes include 5-Amino-9-diethyliminobenzo(a)phenoxazonium Perchlorate; 7-Amino-4-methylcarbostyryl; 7-Amino-4-methylcoumarin; 7-Amino-4-trifluoromethylcoumarin; 3-(2′-Benzimidazolyl)-7-N,N-diethylaminocoumarin; 3-(2′-Benzothiazolyl)-7-diethylaminocoumarin; 2-(4-Biphenylyl)-5-(4-t-butylphenyl)-1,3,4-oxadiazole; 2-(4-Biphenylyl)-5-phenyl-1,3,4-oxadiazole; 2-(4-Biphenyl)-6-phenylbenzoxazole-1,3; 2,5-Bis-(4-biphenylyl)-1,3,4-oxadiazole; 2,5-Bis-(4-biphenylyl)-oxazole; 4,4′″-Bis-(2-butyloctyloxy)-p-quaterphenyl; p-Bis(o-methylstyryl)-benzene; 5,9-Diaminobenzo(a)phenoxazonium Perchlorate; 4-Dicyanomethylene-2-methyl-6-(p-dimethylaminostyryl)-4H-pyran; 1,1′-Diethyl-2,2′-carbocyanine Iodide; 1,1′-Diethyl-4,4′-carbocyanine Iodide; 3,3′-Diethyl-4,4′,5,5′-dibenzothiatricarbocyanine Iodide; 1,1′-Diethyl-4,4′-dicarbocyanine Iodide; 1,1′-Diethyl-2,2′-dicarbocyanine Iodide; 3,3′-Diethyl-9,11-neopentylenethiatricarbocyanine Iodide; 1,3′-Diethyl-4,2′-quinolyloxacarbocyanine Iodide; 1,3′-Diethyl-4,2′-quinolylthiacarbocyanine Iodide; 3-Diethylamino-7-diethyliminophenoxazonium Perchlorate; 7-Diethylamino-4-methylcoumarin; 7-Diethylamino-4-trifluoromethylcoumarin; 7-Diethylaminocoumarin; 3,3′-Diethyloxadicarbocyanine Iodide; 3,3′-Diethylthiacarbocyanine Iodide; 3,3′-Diethylthiadicarbocyanine Iodide; 3,3′-Diethylthiatricarbocyanine Iodide; 4,6-Dimethyl-7-ethylaminocoumarin; 2,2′″-Dimethyl-p-quaterphenyl; 2,2″-Dimethyl-p-terphenyl; 7-Dimethylamino-1-methyl-4-methoxy-8-azaquinolone-2; 7-Dimethylamino-4-methylquinolone-2; 7-Dimethylamino-4-trifluoromethylcoumarin; 2-(4-(4-Dimethylaminophenyl)-1,3-butadienyl)-3-ethylbenzothiazolium Perchlorate; 2-(6-(p-Dimethylaminophenyl)-2,4-neopentylene-1,3,5-hexatrienyl)-3-methylbenzothiazolium Perchlorate; 2-(4-(p-Dimethylaminophenyl)-1,3-butadienyl)-1,3,3-trimethyl-3H-indolium Perchlorate; 3,3′-Dimethyloxatricarbocyanine Iodide; 2,5-Diphenylfuran; 2,5-Diphenyloxazole; 4,4′-Diphenylstilbene; 1-Ethyl-4-(4-(p-Dimethylaminophenyl)-1,3-butadienyl)-pyridinium Perchlorate; 1-Ethyl-2-(4-(p-Dimethylaminophenyl)-1,3-butadienyl)-pyridinium Perchlorate; 1-Ethyl-4-(4-(p-Dimethylaminophenyl)-1,3-butadienyl)-quinolium Perchlorate; 3-Ethylamino-7-ethylimino-2,8-dimethylphenoxazin-5-ium Perchlorate; 9-Ethylamino-5-ethylamino-10-methyl-5H-benzo(a)phenoxazonium Perchlorate; 7-Ethylamino-6-methyl-4-trifluoromethylcoumarin; 7-Ethylamino-4-trifluoromethylcoumarin; 1,1′,3,3,3′,3′-Hexamethyl-4,4′,5,5′-dibenzo-2,2′-indotricarboccyanine Iodide; 1,1′,3,3,3′,3′-Hexamethylindodicarbocyanine Iodide; 1,1′,3,3,3′,3′-Hexamethylindotricarbocyanine Iodide; 2-Methyl-5-t-butyl-p-quaterphenyl; 3-(2′-N-Methylbenzimidazolyl)-7-N,N-diethylaminocoumarin; 2-(1-Naphthyl)-5-phenyloxazole; 2,2′-p-Phenylen-bis(5-phenyloxazole); 3,5,3′″″,5′″″-Tetra-t-butyl-p-sexiphenyl; 3,5,3″″,5″″-Tetra-t-butyl-p-quinquephenyl; 2,3,5,6-1H,4H-Tetrahydro-9-acetylquinolizino-<9,9a,1-gh>coumarin; 2,3,5,6-1H,4H-Tetrahydro-9-carboethoxyquinolizino-<9,9a,1-gh>coumarin; 2,3,5,6-1H,4H-Tetrahydro-8-methylquinolizino-<9,9a,1->coumarin; 2,3,5,6-1H,4H-Tetrahydro-9-(3-pyridyl)-quinolizino-<9,9a,1-gh>coumarin; 2,3,5,6-1H,4H-Tetrahydro-8-trifluoromethylquinolizino-<9,9a,1-gh>coumarin; 2,3,5,6-1H,4H-Tetrahydroquinolizino-<9,9a,1-gh>coumarin; 3,3′,2″,3′″-Tetramethyl-p-quaterphenyl; 2,5,2″″,5″″-Tetramethyl-p-quinquephenyl; P-terphenyl; P-quaterphenyl; Nile Red; Rhodamine 700; Oxazine 750; Rhodamine 800; IR 125; IR 144; IR 140; IR 132; IR 26; IR 5; Diphenylhexatriene; Diphenylbutadiene; Tetraphenylbutadiene; Naphthalene; Anthracene; Pyrene; Chrysene; Rubrene; Coronene; Phenanthrene; Fluorene; Aluminum phthalocyanine; Platinum octaethylporphyrin; and the like.

Other examples of fluorescent dyes are listed below:

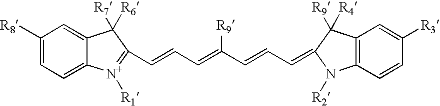

wherein R₁′, R₂′, R₃′, R₄′, R₅′, R₆′, R₇′, R₈′, and R₉′ are each independently selected from the group consisting of H, halogen, alkyl of from 1 to 20 carbon atoms, cycloalkyl of from 3 to 8 carbon atoms, heterocycloalkyl of from 2 to 8 carbon atoms, aryl or heteroaryl of from 4 to 20 carbon atoms, alkoxy, thioether, C(=Z)R, C(=Z)N(R)₂, COCl, amino, CN, nitro, oxiranyl, and glycidyl; wherein Z is O or NR, and R is an aryl or heteroary of from 4 to 20 carbon atoms, or an alkyl, alkynyl, or alkenyl of from 1 to 20 carbon atoms (preferably Z is O), and at least one of R₁′, R₂′, R₃′, R₄′, R₅′, R₆′, R₇′, R₈′, and R₉′ can be further reacted to form a silane moiety.

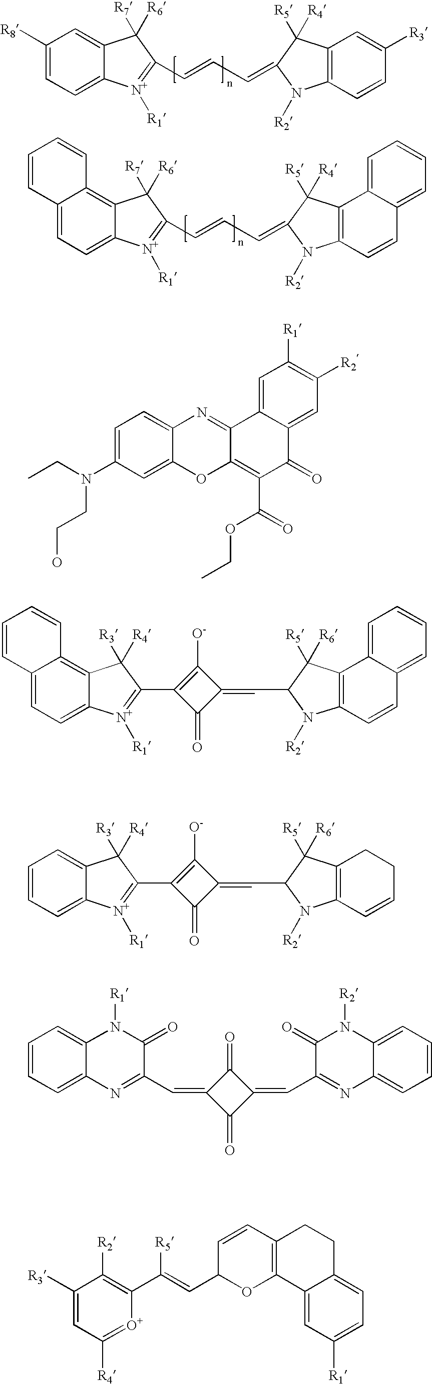

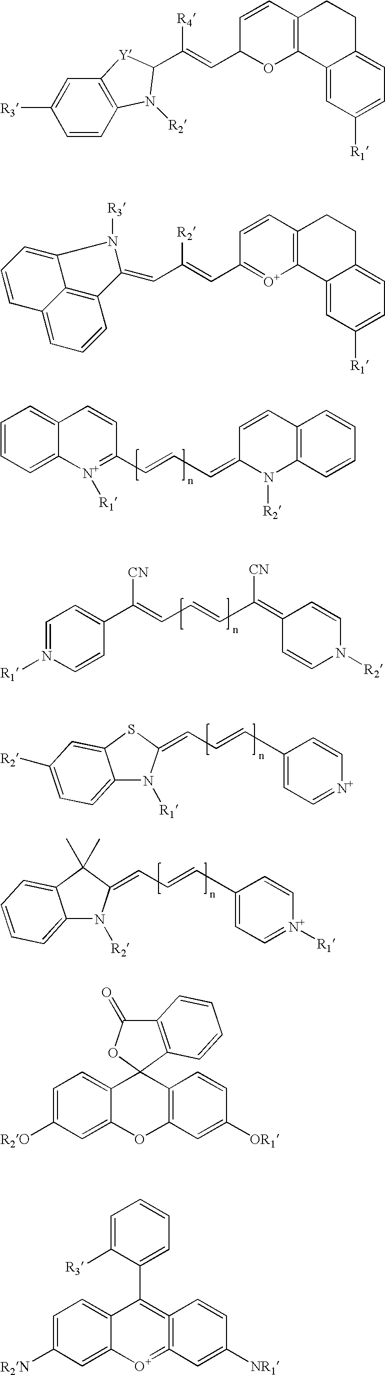

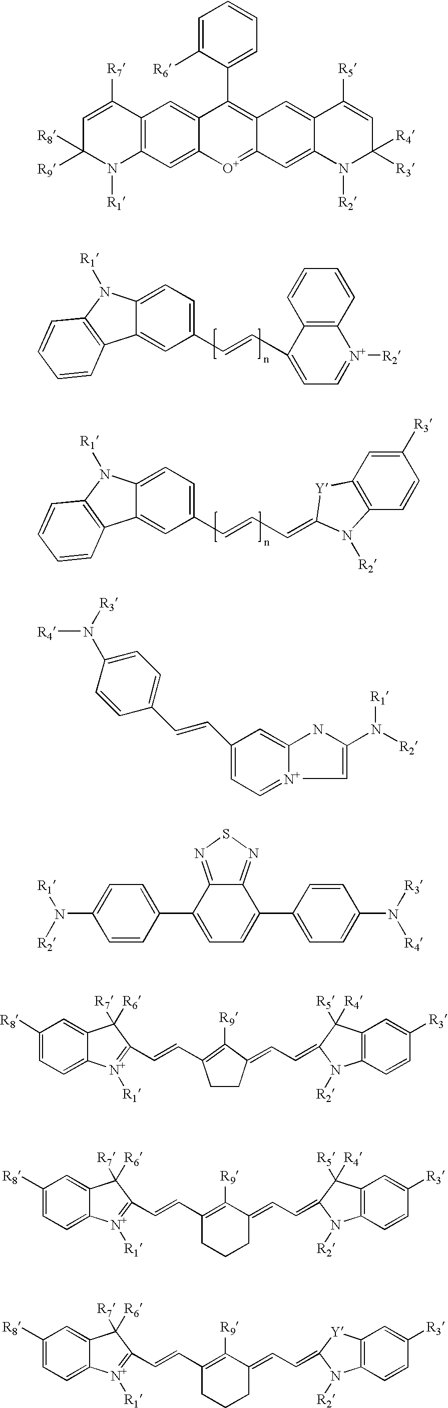

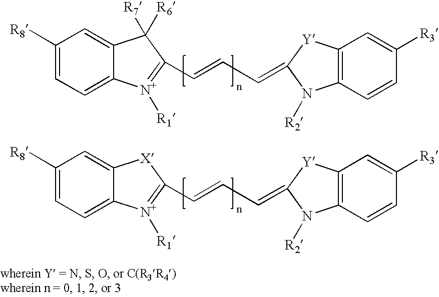

wherein X′ and Y′ are independently S, O, C(R₄′R₅′), or N

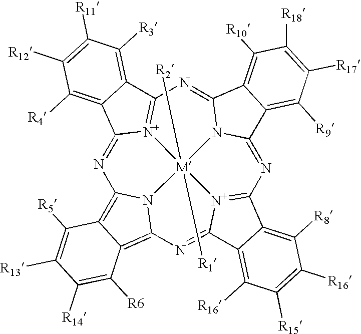

### Wherein M′=Silicon, Magnesium, Aluminum, or Germanium; wherein R₁₀′, R₁₁′, R₁₂′, R₁₃′, R₁₄′, R₁₅′, R₁₆′, R₁₇′, and R₁₈′ are defined as R₁′, R₂′, R₃′, R₄′, R₅′, R₆′, R₇′, R₈′, and R₉′ above.

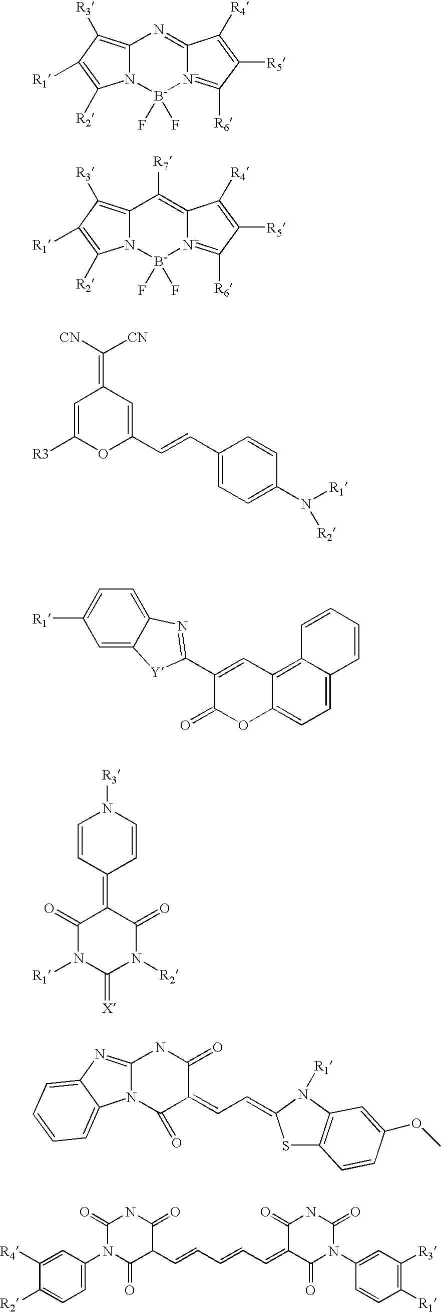

The present nanoparticles can be useful as a carrier for carrying a biological, pharmaceutical or diagnostic component. Specifically, the nanoparticle used as a carrier does not necessarily encapsulate a specific therapeutic or an imaging component, but rather serve as a carrier for the biological, pharmaceutical or diagnostic components, such as therapeutic agents, diagnostic agents, dyes or radiographic contrast agents. The term “diagnostic agent” includes components that can act as contrast agents and thereby produce a detectable indicating signal in the host mammal. The detectable indicating signal may be gamma-emitting, radioactive, echogenic, fluoroscopic or physiological signals, or the like. The term “biomedical agent”, as used herein, includes biologically active substances which are effective in the treatment of a physiological disorder, pharmaceuticals, enzymes, hormones, steroids, recombinant products, and the like. Exemplary therapeutic agents are antibiotics, thrombolytic enzymes such as urokinase or streptokinase, insulin, growth hormone, chemotherapeutics such as adriamycin and antiviral agents such as interferon and acyclovir. Upon enzymatic degradation, such as by a protease or a hydrolase, the therapeutic agents can be released over a period of time.

A variety of drugs with diverse characteristics, including genes and proteins, can be incorporated into the core by engineering the structure of the core-forming segment of the block copolymer so that one can expect a sufficiently strong interaction with drug molecules. Compared to surfactant micelles, polymeric micelles are generally more stable, with a remarkably lowered critical micellar concentration (CMC), and have a slower rate of dissociation, allowing retention of loaded drugs for a longer period of time and, eventually, achieving higher accumulation of a drug at the target site. Furthermore, polymeric micelles have a size range of several tens of nanometers (mesoscopic size range) with a considerably narrow distribution. This narrow size range is similar to that of viruses and lipoproteins, natural mesoscopic-scaled vehicle systems, and is certainly a crucial factor in determining their body disposition, especially when an enhanced permeation retention effect (EPR effect) is involved.

The distribution of drug-loaded polymeric micelles in the body may be determined mainly by their size and surface properties and these are less affected by the properties of loaded drugs if they are embedded in the inner core of the micelles. In this regard, the design of the size and surface properties of polymeric micelles have crucial importance in achieving modulated drug delivery with remarkable efficacy.

Functionalization of the outer surface of the polymeric micelle to modify its physicochemical and biological properties is of great value from the standpoint of designing micellar carrier systems for receptor-mediated drug delivery. This can be accomplished in a regulated fashion by constructing micelles from a variety of end-functionalized block copolymers. Indeed, polymeric micelles that have sugars and peptides on their periphery have recently been prepared, as will be described in the following sections, so that their utility in the field of gene and drug delivery can be explored.

Included within the scope of the invention are compositions comprising the polymer networks of the current invention and a suitable targeting molecule. As used herein, the term “targeting molecule” refers to any molecule, atom, or ion linked to the polymer networks of the current invention that enhance binding, transport, accumulation, residence time, bioavailability or modify biological activity of the polymer networks or biologically active compositions of the current invention in the body or cell. The targeting molecule will frequently comprise an antibody, fragment of antibody or chimeric antibody molecules typically with specificity for a certain cell surface antigen. It could also be, for instance, a hormone having a specific interaction with a cell surface receptor, or a drug having a cell surface receptor. For example, glycolipids could serve to target a polysaccharide receptor. It could also be, for instance, enzymes, lectins, and polysaccharides. Low molecular mass ligands, such as folic acid and derivatives thereof are also useful in the context of the current invention. The targeting molecules can also be polynucleotide, polypeptide, peptidomimetic, carbohydrates including polysaccharides, derivatives thereof or other chemical entities obtained by means of combinatorial chemistry and biology. Targeting molecules can be used to facilitate intracellular transport of the nanoparticles of the invention, for instance transport to the nucleus, by using, for example, fusogenic peptides as targeting molecules described by Soukchareun et al., Bioconjugate Chem., 6, 43, (1995) or Arar et al., Bioconjugate Chem., 6, 43 (1995), caryotypic peptides, or other biospecific groups providing site-directed transport into a cell (in particular, exit from endosomic compartments into cytoplasm, or delivery to the nucleus).

The described composition can further comprise a biological, pharmaceutical or diagnostic component that includes a targeting moiety that recognizes the specific target cell. Recognition and binding of a cell surface receptor through a targeting moiety associated with a described nanoparticle used as a carrier can be a feature of the described compositions. For purposes of the present invention, a compound carried by the nanoparticle may be referred to as a “carried” compound. For example, the biological, pharmaceutical or diagnostic component that includes a targeting moiety that recognizes the specific target cell described above is a “carried” compound. This feature takes advantage of the understanding that a cell surface binding event is often the initiating step in a cellular cascade leading to a range of events, notably receptor-mediated endocytosis. The term “Receptor Mediated Endocytosis” (“RME”) generally describes a mechanism by which, catalyzed by the binding of a ligand to a receptor disposed on the surface of a cell, a receptor-bound ligand is internalized within a cell. Many proteins and other structures enter cells via receptor mediated endocytosis, including insulin, epidermal growth factor, growth hormone, thyroid stimulating hormone, nerve growth factor, calcitonin, glucagon and many others.

Receptor Mediated Endocytosis affords a convenient mechanism for transporting a described nanoparticle, possibly containing other biological, pharmaceutical or diagnostic components, to the interior of a cell. In RME, the binding of a ligand by a receptor disposed on the surface of a cell can initiate an intracellular signal, which can include an endocytosis response. Thus, a nanoparticle used as a carrier with an associated targeting moiety, can bind on the surface of a cell and subsequently be invaginated and internalized within the cell. A representative, but non-limiting, list of moieties that can be employed as targeting agents useful with the present compositions includes proteins, peptides, aptomers, small organic molecules, toxins, diptheria toxin, pseudomonas toxin, cholera toxin, ricin, concanavalin A, Rous sarcoma virus, Semliki forest virus, vesicular stomatitis virus, adenovirus, transferrin, low density lipoprotein, transcobalamin, yolk proteins, epidermal growth factor, growth hormone, thyroid stimulating hormone, nerve growth factor, calcitonin, glucagon, prolactin, luteinizing hormone, thyroid hormone, platelet derived growth factor, interferon, catecholamines, peptidomimetrics, glycolipids, glycoproteins and polysacchorides. Homologs or fragments of the presented moieties can also be employed. These targeting moieties can be associated with a nanoparticle and be used to direct the nanoparticle to a target cell, where it can subsequently be internalized. There is no requirement that the entire moiety be used as a targeting moiety. Smaller fragments of these moieties known to interact with a specific receptor or other structure can also be used as a targeting moiety.

An antibody or an antibody fragment represents a class of most universally used targeting moiety that can be utilized to enhance the uptake of nanoparticles into a cell. Antibodies may be prepared by any of a variety of techniques known to those of ordinary skill in the art. See, e.g., Harlow and Lane, Antibodies: A Laboratory Manual, Cold Spring Harbor Laboratory, 1988. Antibodies can be produced by cell culture techniques, including the generation of monoclonal antibodies or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). A superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described (Winter et al. (1991) Nature 349:293-299; Lobuglio et al. (1989) Proc. Nat. Acad. Sci. USA 86:4220-4224. These “humanized” molecules are designed to minimize unwanted immunological response toward rodent antihuman antibody molecules that limits the duration and effectiveness of therapeutic applications of those moieties in human recipients.

Vitamins and other essential minerals and nutrients can be utilized as targeting moiety to enhance the uptake of nanoparticle by a cell. In particular, a vitamin ligand can be selected from the group consisting of folate, folate receptor-binding analogs of folate, and other folate receptor-binding ligands, biotin, biotin receptor-binding analogs of biotin and other biotin receptor-binding ligands, riboflavin, riboflavin receptor-binding analogs of riboflavin and other riboflavin receptor-binding ligands, and thiamin, thiamin receptor-binding analogs of thiamin and other thiamin receptor- binding ligands. Additional nutrients believed to trigger receptor mediated endocytosis, and thus also having application in accordance with the presently disclosed method, are camitine, inositol, lipoic acid, niacin, pantothenic acid, pyridoxal, and ascorbic acid, and the lipid soluble vitamins A, D, E and K. Furthermore, any of the “immunoliposomes” (liposomes having an antibody linked to the surface of the liposome) described in the prior art are suitable for use with the described compositions.

Since not all natural cell membranes possess biologically active biotin or folate receptors, use of the described compositions in-vitro on a particular cell line can involve altering or otherwise modifying that cell line first to ensure the presence of biologically active biotin or folate receptors. Thus, the number of biotin or folate receptors on a cell membrane can be increased by growing a cell line on biotin or folate deficient substrates to promote biotin and folate receptor production, or by expression of an inserted foreign gene for the protein or apoprotein corresponding to the biotin or folate receptor.

RME is not the exclusive method by which the described nanoparticle can be translocated into a cell. Other methods of uptake that can be exploited by attaching the appropriate entity to a nanoparticle include the advantageous use of membrane pores. Phagocytotic and pinocytotic mechanisms also offer advantageous mechanisms by which a nanoparticle can be internalized inside a cell.

The recognition moiety can further comprise a sequence that is subject to enzymatic or electrochemical cleavage. The recognition moiety can thus comprise a sequence that is susceptible to cleavage by enzymes present at various locations inside a cell, such as proteases or restriction endonucleases (e.g. DNAse or RNAse).

A cell surface recognition sequence is not a requirement. Thus, although a cell surface receptor targeting moiety can be useful for targeting a given cell type, or for inducing the association of a described nanoparticle with a cell surface, there is no requirement that a cell surface receptor targeting moiety be present on the surface of a nanoparticle.

To assemble the biological, pharmaceutical or diagnostic components to a described nanoparticle used as a carrier, the components can be associated with the nanoparticle carrier through a linkage. By “associated with”, it is meant that the component is carried by the nanoparticle. The component can be dissolved and incorporated in the nanoparticle non-covalently.

Generally, any manner of forming a linkage between a biological, pharmaceutical or diagnostic component of interest and a nanoparticle used as a carrier can be utilized. This can include covalent, ionic, or hydrogen bonding of the ligand to the exogenous molecule, either directly or indirectly via a linking group. The linkage is typically formed by covalent bonding of the biological, pharmaceutical or diagnostic component to the nanoparticle used as a carrier through the formation of amide, ester or imino bonds between acid, aldehyde, hydroxy, amino, or hydrazo groups on the respective components of the complex. Art-recognized biologically labile covalent linkages such as imino bonds and so-called “active” esters having the linkage —COOCH, —O—O— or —COOCH are preferred. The biological, pharmaceutical or diagnostic component of interest may be attached to the pre-formed nanoparticle or alternately the component of interest may be pre-attached to a polymerizeable unit and polymerized directly into the nanoparticle during the nanoparticle preparation. Hydrogen bonding, e.g that occurring between complementary strands of nucleic acids, can also be used for linkage formation.

In a preferred embodiment of this invention, the biological, pharmaceutical or diagnostic component of interest is attached to the nanoparticle by reaction with a reactive chemical unit at the terminus of the highly hydrophilic macromonomer units. The reactive chemical unit includes but are not limited to thiols, chloromethyl, bromomethyl, amines, carboxylic acid or activated ester, vinylsulfonyls, aldehydes, epoxies, hydrazides, succinimidyl esters, maleimides, a-halo carbonyl moieties (such as iodoacetyls), isocyanates, isothiocyanates, and aziridines. Preferably this reactive chemical unit is a thiol, a carboxylic acid, an amine, or an activated ester. Most preferably, this attachment occurs via a linking polymer.

The linking polymer may be used in both the acylation and alkylation approaches and is compatible with aqueous and organic solvent systems, so that there is more flexibility in reacting with useful groups and the desired products are more stable in an aqueous environment, such as a physiological environment. The linking polymer has a poly (ethylene glycol) backbone structure which contains at least two reactive groups, one at each end. The poly (ethylene glycol) macromonomer backbone contains a radical polymerizeable group at one end. This group can be, but is not necessarily limited to a methacrylate, acrylate, acrylamide, methacrylamide, styrenic, allyl, vinyl, maleimide, or maleate ester. The poly (ethylene glycol) macromonomer backbone additionally contains a reactive chemical functionality at the other end which can serve as an attachment point for other chemical units, such as quenchers or antibodies. This chemical functionality may be, but is not limited to thiols, carboxylic acids, primary or secondary amines, chloromethyl, bromomethyl, vinylsulfonyls, aldehydes, epoxies, hydrazides, succinimidyl esters, maleimides, a-halo carbonyl moieties (such as iodoacetyls), isocyanates, isothiocyanates, and aziridines. Preferably, these functionalities will be carboxylic acids, primary amines, thiols, maleimides, vinylsulfonyls, or secondary amines. More preferably, one of the reactive groups is an acrylate, a cyanoacrylate, a styrene, or a methacrylate which are useful for free radical polymerization to form copolymers. Most preferably, one of the reactive groups is an acrylate, a cyanoacrylate, or a methacrylate which is useful for reacting with thiols through Michael addition to generate the other reactive group. The other reactive group is useful for conjugation to contrast agents, dyes, proteins, amino acids, peptides, antibodies, bioligands, therapeutic agents and enzyme inhibitors. The linking polymer may be branched or unbranched. Preferably, for therapeutic use of the end-product preparation, the linking polymer will be pharmaceutically acceptable. The poly (ethylene glycol) macromonomer may have a molecular weight of between 300 and 10,000, preferably between 500 and 5000.

A particularly preferred water-soluble linking polymer for use herein is a poly(ethylene glycol) derivative of Formula A. The poly(ethylene glycol) (PEG) backbone of the linking polymer is a hydrophilic, biocompatible and non-toxic polymer of general formula H(OCH₂CH₂)ₙOH, wherein n>4.

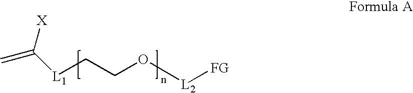

Wherein X is selected from the group consisting of H, halogen, CN, CF₃, alkyl of from 1 to 20 carbon atoms, alkenyl or alkynyl of 2 to 10 carbon atoms, cycloalkyl of from 3 to 8 carbon atoms, heterocycloalkyl of from 2 to 8 carbon atoms, aryl of from 6 to 20 carbon atoms, heteroaryl of from 5 to 20 carbon atoms; preferably X is H or alkyl of from 1 to 20 carbon atoms, and more preferably X is CH3 or H; L₁ and L₂ are direct bonds or linking groups or spacers, FG is a functional group and include but are not limited to thiols, amines, carboxylic acid or activated ester, vinylsulfonyls, aldehydes, epoxies, hydrazides, succinimidyl esters, chloromethyl, bromomethyl, maleimides, a-halo carbonyl moieties (such as iodoacetyls), isocyanates, isothiocyanates, and aziridines, preferably FG is a thiol, a carboxylic acid, an amine, or an activated ester, and n is greater than 4 and less than 1000. More preferably, the water-soluble liking polymer is a poly(ethylene glycol) (PEG) derivative of Formula I and II

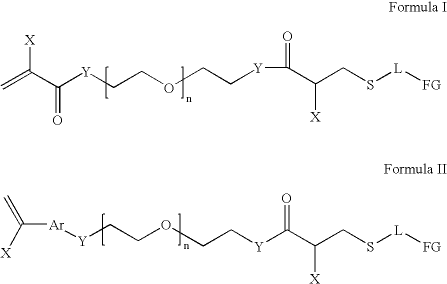

wherein X═CH3 or H, Y═O, NR, or S, L is a linking group or spacer, FG is a functional group, Ar is an aryl or heteroaryl group, and n is greater than 4 and less than 1000. Most preferably, X═CH3, Y═O, NR, L is alkyl or aryl and FG is NH2, SH, or COOH, and n is between 6 and 500 or between 10 and 200. Most preferably, n=16.

The following is a list of preferred linking polymers, but is not intended to an exhaustive and complete list of all linking polymers according to the present invention:

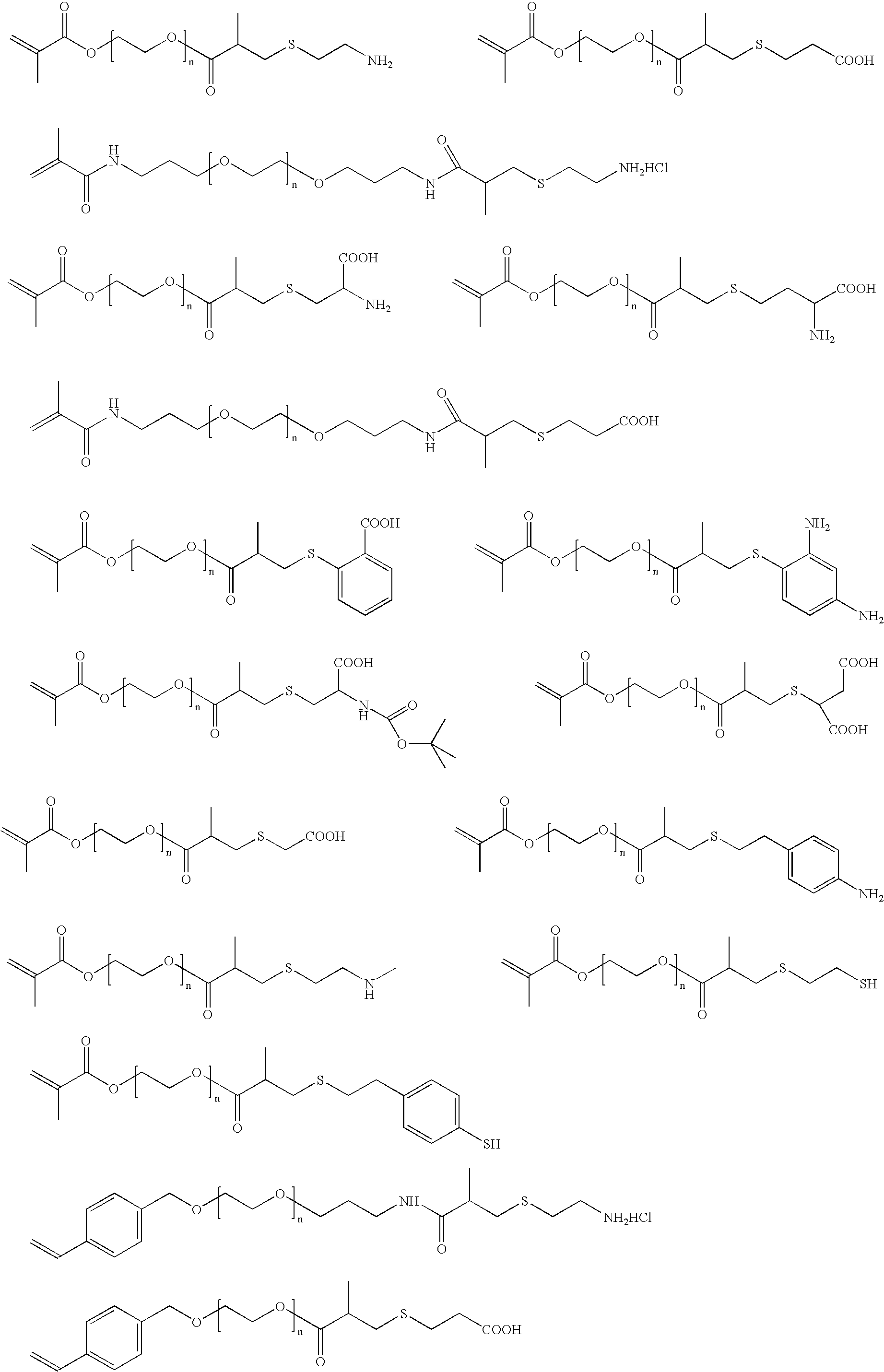

Suitable atom transfer radical polymerization initiators of the present invention are represented by Formula III

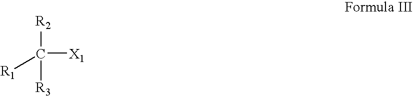

### Wherein

X₁ is selected from the group consisting of Cl, Br, I, OR, SR, SeR, OC(═O)R, OP(═O)R, OP(═O)(OR)₂, OP(═O)OR, O—N(R)₂, and S—C(═S)N(R)₂, wherein R is an aryl or heteroary of from 4 to 20 carbon atoms, or an alkyl, alkynyl, or alkenyl of from 1 to 20 carbon atoms; preferably, X₁ is Cl or Br;

R₁, R₂, and R₃ are each independently selected from the group consisting of H (preferably no more than one of R₁, R₂, and R₃ is H), halogen, alkyl of from 1 to 20 carbon atoms, cycloalkyl of from 3 to 8 carbon atoms, heterocycloalkyl of from 2 to 8 carbon atoms, aryl or heteroaryl of from 4 to 20 carbon atoms, C(=Z)R, C(=Z)N(R)₂, COCl, OH (preferably only one of R₁, R₂, and R₃ is OH), CN, oxiranyl, and glycidyl; wherein Z is O or NR (preferably Z is O) and R is an aryl or heteroary of from 4 to 20 carbon atoms, or an alkyl, alkynyl, or alkenyl of from 1 to 20 carbon atoms. R₁, R₂, and R₃ can also contain reactive functional group such as NH₂, SH, OH, CH₂Cl and COOH so that the functional group will be at the terminus of the copolymer chain. These functional groups can be used for crosslinking if on the hydrophobic end of the copolymer or used for forming a linkage between a biological, pharmaceutical or diagnostic component of interest and the nanoparticle if on the hydrophilic end of the copolymer after the micelle formation.

Examples of suitable initiators include 1-phenylethyl chloride, 1-phenylethyl bromide, chloroform, carbon tetrachloride, 2-chloropropionitrile, 2-chloropropionic acid, 2-bromopropionic acid, 2-bromoisobutyric acid, 2-chloroisobutyric acid, methyl 2-chloropropionate, ethyl 2-chloropropionate, methyl 2-bromopropionate, ethyl 2-bromoisobutyrate, α,α′-dichloroxylene, α,α′-dibromoxylene, hexakis(α-bromomethyl)benzene, and compounds represented by the following structures

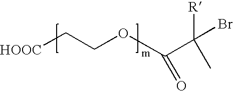

Compound 1, m=1, R′=Me
Compound 2, m=2, R′=Me
Compound 3, m=3, R′=Me
Compound 4, m=4, R′=Me
Compound 5, m=4, R′═H
Compound 6, m=2, R′═H
Compound 7, m=3, R′═H
Compound 8, m=4, R′═H

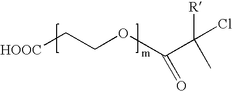

Compound 9, m=1, R′=Me
Compound 10, m=2, R′=Me
Compound 11, m=3, R′=Me
Compound 12, m=4, R′=Me
Compound 13, m=4, R′═H
Compound 14, m=2, R′═H
Compound 15, m=3, R′═H
Compound 16, m=4, R′═H

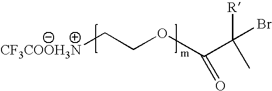

Compound 17, m=1, R′=Me
Compound 18, m=2, R′=Me
Compound 19, m=3, R′=Me
Compound 20, m=4, R′=Me
Compound 21, m=1, R′═H
Compound 22, m=2, R′═H
Compound 23, m=3, R′═H
Compound 24, m=4, R′═H

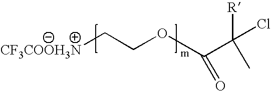

Compound 25, m=1, R′=Me
Compound 26, m=2, R′=Me
Compound 27, m=3, R′=Me
Compound 28, m=4, R′=Me
Compound 29, m=4, R′═H
Compound 30, m=2, R′═H
Compound 31, m=3, R′═H
Compound 32, m=4, R′═H

Other suitable atom transfer radical polymerization initiators of the present invention are macroinitiators incorporating poly(ethylene oxide) segments, represented by Formula IV

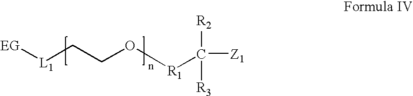

### Wherein L₁, X₁, R₁, R₂, R₃ and n are defined as above, and EG is an end group that include reactive functional group FG as defined above, and other non-reactive groups such as H, alkyl, cycloalkyl, alkoxy, aryl, or heteroaryl.

Preferred macroinitiators are represented by the following structures

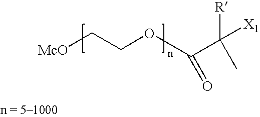

Compound 33, R′═H, X₁═Cl
Compound 34, R′═H, X₁═Br
Compound 35, R′=Me, X₁═Cl
Compound 36, R′=Me, X₁═Br

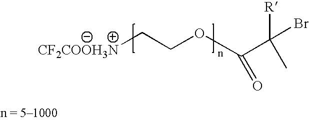

Compound 37, R′═H, X₁═Cl
Compound 38, R′═H, X₁═Br
Compound 39, R′=Me, X₁═Cl
Compound 40, R′=Me, X₁=Br

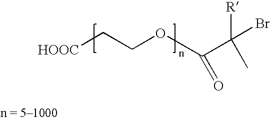

Compound 41, R′═H, X₁═Cl
Compound 42, R′═H, X₁═Br
Compound 43, R′=Me, X₁═Cl
Compound 44, R′=Me, X₁=Br

Any transition metal compound which can participate in a redox cycle with the initiator and dormant polymer chain, but which does not form a direct carbon-metal bond with the polymer chain, is suitable for use in the present invention. Preferred transition metal compounds are those of the formula Mₜ^{k+}X′ₖ. wherein Mₜ^{k+} is selected from the group consisting of Cu¹⁺, Cu²⁺, Fe²⁺, Fe³⁺, Ru²⁺, Ru³⁺, Cr³⁺, Cr²⁺, Mo¹⁺, Mo⁰, Mo²⁺, Mo³⁺, W²⁻, W³⁺, Rh³⁺, Rh⁴⁺, Co¹⁻, Co²⁺, Re²⁺, Re²⁺, Re³⁺, Ni¹⁺, Ni⁰, Mn³⁻, Mn⁴⁺, V²⁺, V³⁺, Zn¹⁺, Zn²⁺, Au¹⁺, Au²⁺, Ag¹⁺, and Ag²⁺; X′ is selected from the group consisting of halogen, CN, alkoxy of from 1 to 6 carbon atoms, triflate, hexafluorophosphate, methanesulfonate, arylsulfonate (preferably benzenesulfonate or toluenesulfonate), SeR, RCOO, phosphate, sulfate, and hydrogenphosphate; and k is the formal charge on the metal, and k is an integer from 0 to 7.

Suitable ligands for use in atom transfer radical polymerization in the present invention include ligands having one or more reactive oxygen, nitrogen, phosphorus, and/or sulfur atoms which can coordinate to the transition metal through a σ-bond; ligands containing two or more carbon atoms which can coordinate to the transition metal through a π-bond, and ligands which can coordinate to the transition metal through a μ-bond or a η-bond. Also included as suitable ligands in the present invention are carbon monoxide, porphyrins and porphycenes. Other suitable ligands include carbon-based ligands such as arenes and cycopentadienes. Preferred ligands include pyridines, bipyridines, acetonitrile, (RO)₃P and P(R)₃, wherein R is an aryl or heteroary of from 4 to 20 carbon atoms, or an alkyl, alkynyl, or alkenyl of from 1 to 20 carbon atoms, 1,10-phenanthroline, porphyrin, cryptands and crown ether such as 18-crown-6, and multidentate ligands such as pentamethyldiethylenetriamine (PMDETA) and tris(dimethylaminoethyl)amine (Me₆TREN), Most preferred ligands are bipyridines and (RO)₃P as above, pentamethyldiethylenetriamine (PMDETA) and tris(dimethylaminoethyl)amine (Me₆TREN).

Atom transfer radical polymerization can be conducted in the absence of solvent (bulk polymerization) or in the presence of solvent. Suitable solvents include ethers such as diethyl ether, ethyl propyl ether, dipropyl ether, methyl t-butyl ether, di-t-butyl ether, glyme, and diglyme; cyclic ethers such as dioxane and tetrahydrofuran (THF), C₅-C₁₀ alkanes, C₅-C₈ cycloalkanes, aromatic hydrocarbons such as benzene, toluene, xylenes, and anisole; halogenated hydrocarbons such as methylene chloride, 1,2-dichloroethane, chlorobenzenes, dichlorobenzenes, and fluorobenzene; acetonitrile, dimethylformamide, alcohols such as methanol, ethanol, isopropanol, and water. Preferably, the solvents suitable for polymerization are toluene, xylenes, dichlorobenzens, chlorobenzenes, methanol, and anisole.

Useful monomers for the hydrophobic components of the present invention include but are not limited to α,β-ethylenically unsaturated monomers such as styrenics, acrylamides, and (meth)acrylates, lactones, lactams, lactic acid, and amino acids. Preferably, the hydrophobic components derived from styrenics, (meth)acrylamides, and (meth)acrylates. More preferably, the hydrophobic components derived from styrenics and (meth)acrylates containing cross-linkable alkoxy silane or amino silane moiety represented by Formula V and VI

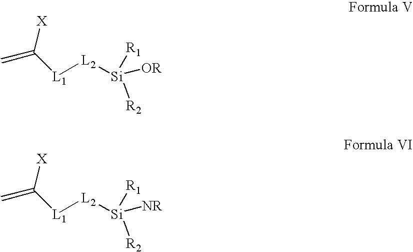

### Wherein X, L₁, L₂, R₁, R₂, and R are defined above.

Preferably, the monomers for the hydrophobic components of the present invention include, but are not limited to, the following structures:

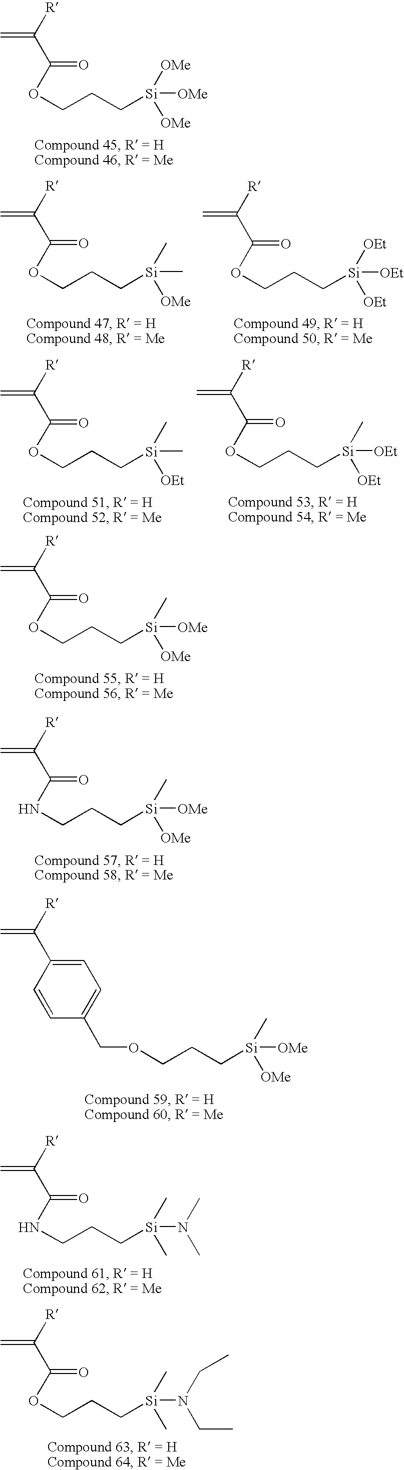

Both hydrophilic and hydrophobic segment of the copolymer can be derived from more than one monomer as long as each block remains sufficiently hydrophilic or hydrophobic. The properties of both segments thus can be tailored by copolymerization with other monomers.

During the preparation of micellar nanoparticle, auxiliary crosslinking agents can also be included in the micellar core. Examples of auxiliary crosslinking agents include polyfunctional α,β-ethylenically unsaturated moieties such as styrenics, (meth)acrylates, (meth)acrylamides, polyfunctional epoxide, polyfunctional aziridenes, or polyfunctional silanes. Preferably, the auxiliary crosslinking agents are polyfunctional silanes such as tetraethoxysilane, 1,2-bis(triethoxysily)octane, 1,2-bis(trimethoxysily)decane, octadecyltriethoxy silane.

After a sufficiently pure nanoparticle, preferably comprising a nanoparticle with a biological, pharmaceutical or diagnostic component, has been prepared, it might be desirable to prepare the nanoparticle in a pharmaceutical composition that can be administered to a subject or sample. Preferred administration techniques include parenteral administration, intravenous administration and infusion directly into any desired target tissue, including but not limited to a solid tumor or other neoplastic tissue. Purification can be achieved by employing a final purification step, which dissolves the nanoparticle in a medium comprising a suitable pharmaceutical composition. Suitable pharmaceutical compositions generally comprise an amount of the desired nanoparticle with active agent in accordance with the dosage information (which is determined on a case-by-case basis). The described nanoparticles are admixed with an acceptable pharmaceutical diluent or excipient, such as a sterile aqueous solution, to give an appropriate final concentration. Such formulations can typically include buffers such as phosphate buffered saline (PBS), or additional additives such as pharmaceutical excipients, stabilizing agents such as bovine serum albumin (BSA) or human serum albumin (HSA), or salts such as sodium chloride.

For parenteral administration it is generally desirable to further render such compositions pharmaceutically acceptable by insuring their sterility, non-immunogenicity and non-pyrogenicity. Such techniques are generally well known in the art. Moreover, for human administration, preparations should meet sterility, pyrogenicity, general safety and purity standards as required by FDA Office of Biological Standards. When the described nanoparticle composition is being introduced into cells suspended in a cell culture, it is sufficient to incubate the cells together with the nanoparticle in an appropriate growth media, for example Luria broth (LB) or a suitable cell culture medium. Although other introduction methods are possible, these introduction treatments are preferable and can be performed without regard for the entities present on the surface of a nanoparticle used as a carrier.

Included within the scope of the invention are compositions comprising nanoparticles of the current invention and other suitable imagable moieties. The nature of the imagable moiety depends on the imaging modality utilized in the diagnosis. The imagable moiety must be capable of detection either directly or indirectly in an in vivo diagnostic imaging procedure, for example, moieties which emit or may be caused to emit detectable radiation (e.g. by radioactive decay, fluorescence excitation, spin resonance excitation, etc.), moieties which affect local electromagnetic fields (e.g. paramagnetic, superparamagnetic, ferrimagnetic or ferromagnetic species), moieties which absorb or scatter radiation energy (e.g. chromophores, particles (including gas or liquid containing vesicles), heavy elements and compounds thereof, etc.), and moieties which generate a detectable substance (e.g. gas microbubble generators), etc.

A very wide range of materials detectable by diagnostic imaging modalities is known from the art. Thus for example for ultrasound imaging an echogenic material, or a material capable of generating an echogenic material will normally be selected, for X-ray imaging the imagable moieties will generally be or contain a heavy atom (e.g. of atomic weight 38 or above), for magnetic resonance imaging (MRI) the imagable moieties will either be a non zero nuclear spin isotope (such as ¹⁹F) or a material having unpaired electron spins and hence paramagnetic, superparamagnetic, ferrimagnetic or ferromagnetic properties, for light imaging the imagable moieties will be a light scatterer (eg. a colored or uncolored particle), a light absorber or a light emitter, for magnetometric imaging the imagable moieties will have detectable magnetic properties, for electrical impedance imaging the imagable moieties will affect electrical impedance and for scintigraphy, SPECT, PET etc. the imagable moieties will be a radionuclide.

Examples of the suitable imagable moieties are widely known from the diagnostic imaging literature, eg. magnetic iron oxide particles, gas-containing vesicles, chelated paramagnetic metals (such as Gd, Dy, Mn, Fe etc.). Particularly preferred imagable moieties are: chelated paramagnetic metal ions such as Gd, Dy, Fe, and Mn, especially when chelated by macrocyclic chelant groups (eg. tetraazacyclododecane chelants such as 1,4,7,10-tetraazacyclododecane-N,N′,N″,N′″-tetraacetic acid (DOTA), 1,4,7,10-tetraazacyclododecane-N,N′,N″-triacetic acid (D03A), HP-D03A (10-(2-hydroxypropyl)-1,4,7,10-tetraazacyclododecane-1,4,7triacetic acid) and analogues thereof, or by linker chelant groups such as DTPA (N,N,N′,N″,N″-diethylene-triaminepentaacetic acid (DTPA), DTPA-BMA (N,N,N′,N″,N″-diethylenetriaminepentaacetic acid bismethylamide), DPDP (N,N′-dipyridoxylethylenediamine-N,N′-diacetate-5,5′-bis(phosphate), ethylenediamine-N,N,N′,N′-tetraacetic acid (EDTA), 1-oxa-4,7,10-triazacyclododecane-N,N′,N″-triacetic acid (OTTA), trans(1,2)-cyclohexanodiethylene-triamine-pentaacetic acid (CDTPA), etc; metal radionuclide such as ⁹⁰Y, ^{99m}Tc, ¹¹¹In, ⁴⁷Sc, ⁶⁷Ga, ⁵¹Cr, ^{177m}Sn, ⁶⁷C, ¹⁶⁷Tm, ⁹⁷Ru, ¹⁸⁸Re, ¹⁷⁷Lu, ¹⁹⁹Au, ²⁰³Pb and ¹⁴¹Ce; superparamagnetic iron oxide crystals; chromophores and fluorophores having absorption and/or emission maxima in the range 300-1400 nm, especially 600 nm to 1200 nm, in particular 650 to 1000 nm; vesicles containing fluorinated gases (ie. containing materials in the gas phase at 37° C. which are fluorine containing, eg. SF₆ or perfluorinated C₁₋₆ hydrocarbons or other gases and gas precursors listed in W097/29783); chelated heavy metal cluster ions (eg. W or Mo polyoxoanions or the sulphur or mixed oxygen/sulphur analogs); covalently bonded non-metal atoms which are either high atomic number (eg. iodine) or are radioactive, eg ¹²³I, ¹³¹I, etc. atoms; iodinated compound containing vesicles; etc.

Stated generally, the imagable moieties may be (1) a chelatable metal or polyatomic metal-containing ion (ie. TcO, etc), where the metal is a high atomic number metal (eg. atomic number greater than 37), a paramagentic species (eg. a transition metal or lanthanide), or a radioactive isotope, (2) a covalently bound non-metal species which is an unpaired electron site (eg. an oxygen or carbon in a persistent free radical), a high atomic number non-metal, or a radioisotope, (3) a polyatomic cluster or crystal containing high atomic number atoms, displaying cooperative magnetic behavior (eg. superparamagnetism, ferrimagnetism or ferromagnetism) or containing radionuclides, (4) a gas or a gas precursor (ie. a material or mixture of materials which is gaseous at 37° C.), (5) a chromophore (by which term species which are fluorescent or phosphorescent are included), eg. an inorganic or organic structure, particularly a complexed metal ion or an organic group having an extensive delocalized electron system, or (6) a structure or group having electrical impedance varying characteristics, eg. by virtue of an extensive delocalized electron system. Examples of particular imgable moieties are described in more detail below.

Chelated metal imagable moieties: Metal Radionuclides, Paramagnetic metal ions, Fluorescent metal ions, Heavy metal ions and cluster ions. Preferred metal radionuclides include ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce; Preferred paramagnetic metal ions include ions of transition and lanthanide metals (eg. metals having atomic numbers of 6 to 9, 21-29, 42, 43, 44, or 57-71), in particular ions of Cr, V, Mn, Fe, Co, Ni, Cu, La, Ce, Pr, Nd, Pm, Sm, Eu, Gd, Tb, Dy, Ho, Er, Tm, Yb and Lu, especially of Mn, Cr, Fe, Gd and Dy, more especially Gd. Preferred fluorescent metal ions include lanthanides, in particular La, Ce, Pr,Nd, Pm, Sm, Eu, Gd, Tb, Dy, Ho, Er, Tm, Yb, and Lu.—Eu is especially preferred. Preferred heavy metal-containing imagable moieties may include atoms of Mo, Bi, Si, and W, and in particular may be polyatomic cluster ions (eg. Bi compounds and W and Mo oxides). The metal ions are desirably chelated by chelant groups in particular linear, macrocyclic, terpyridine and N2S2 chelants, such as for example ethylenediamine-N,N,N′,N′-tetraacetic acid (EDTA); N,N,N′,N″,N″-diethylene-triaminepentaacetic acid (DTPA); 1,4,7,10-tetraazacyclododecane-N,N′,N″,N′″-tetraacetic acid (DOTA); 1,4,7,10-tetraazacyclododecane-N,N′,N″-triacetic acid (D03A); 1-oxa-4,7,10-triazacyclododecane-N,N′,N″-triacetic acid (OTTA); trans(1,2)-cyclohexanodiethylene-triamine-pentaacetic acid (CDTPA), TMT (terpyridine-bis(methylenaminetetraacetic acid)

Further examples of suitable chelant groups are disclosed in U.S. Pat. No. 4,647,447, U.S. Pat. No. 5,367,080, U.S. Pat. No. 5,364,613, etc. The imagable moiety may contain one or more such chelant groups, if desired metallated by more than one metal species (eg. so as to provide the imagable moieties detectable in different imaging modalities). Particularly where the metal is non-radioactive, it is preferred that a polychelant moiety is used.

A chelant or chelating group as referred to herein may comprise the residue of one or more of a wide variety of chelating agents that can complex a metal ion or a polyatomic ion (eg. TcO).

As is well known, a chelating agent is a compound containing donor atoms that can combine by coordinate bonding with a metal atom to form a cyclic structure called a chelation complex or chelate. The reside of a suitable chelating agent can be selected from polyphosphates, such as sodium tripolyphosphate and hexametaphosphoric acid; aminocarboxylic acids, such as EDTA (ethylenediaminetetraacetic acid), N-(2-hydroxy)ethylenediaminetriacetic acid, nitrilotriacetic acid, N,N-di(2-hydroxyethyl)glycine, ethylenebis(hydroxyphenylglycine) and diethylenetriamine pentacetic acid; 1,3-diketones, such as acetylacetone, trifluoroacetylacetone, and thenoyltrifluoroacetone; hydroxycarboxylic acids, such as tartaric acid, citric acid, gluconic acid, and 5-sulfosalicyclic acid; polyamines, such as ethylenediamine, diethylenetriamine, triethylenetetraamine, and triaminotriethylamine; aminoalcohols, such as triethanolamine and N-(2-hydroxyethyl) ethylenediamine; aromatic heterocyclic bases, such as 2,21-diimidazole, picoline amine, dipicoline amine and 1,10-phenanthroline; phenols, such as salicylaldehyde, disulfopyrocatechol, and chromotropic acid; aminophenols, such as 8-hydroxyquinoline and oximesulfonic acid; oximes, such as dimethylglyoxime and salicylaldoxime; peptides containing proximal chelating functionality such as polycysteine, polyhistidine, polyaspartic acid, polyglutamic acid, or combinations of such amino acids; Schiff bases, such as disalicylaldehyde 1,2-propylenediimine; tetrapyrroles, such as tetraphenylporphin and phthalocyanine; sulfur compounds, such as toluenedithiol, meso-2,3-dimercaptosuccinic acid, dimercaptopropanol, thioglycolic acid, potassium ethyl xanthate, sodium diethyldithiocarbamate, dithizone, diethyl dithiophosphoric acid, and thiourea; synthetic macrocyclic compounds, such as dibenzo [18-crown-6, (CH₃)₆-[14]-4,11]-diene-N₄, and (2.2.2-cryptate); phosphonic acids, such as nitrilotrimethylene-phosphonic acid, ethylenediaminetetra(methylenephosphonic acid), and hydroxyethylidenediphosphonic acid, or combinations of two or more of the above agents. The residue of a suitable chelating agent preferably comprises a polycarboxylic acid group and preferred examples include: ethylenediamine-N,N,N′,N′-tetraacetic acid (EDTA); N,N,N′,N″,N″-diethylene-triaminepentaacetic acid (DTPA); 1,4,7,10-tetraazacyclododecane-N,N′,N″,N′″-tetraacetic acid (DOTA); 1,4,7,10-tetraazacyclododecaneN,N′,N″-triacetic acid (D03A); 1-oxa-4,7,10-triazacyclododecane-N,N′,N″-triacetic acid (OTTA); trans(1,2)-cyclohexanodiethylene-triamine-pentaacetic acid (CDTPA), other suitable residues of chelating agents comprise proteins modified for the chelation of metals such as technetium and rhenium as described in U.S. Pat. No. 5,078,985, the disclosure of which is hereby incorporated by reference.

Metals can be incorporated into a chelant moiety by any one of three general methods: direct incorporation, template synthesis and/or transmetallation. Direct incorporation is preferred.

Thus it is desirable that the metal ion be easily complexed to the chelating agent, for example, by merely exposing or mixing an aqueous solution of the chelating agent-containing moiety with a metal salt in an aqueous solution preferably having a pH in the range of about 4 to about 11. The salt can be any salt, but preferably the salt is a water soluble salt of the metal such as a halogen salt, and more preferably such salts are selected so as not to interfere with the binding of the metal ion with the chelating agent. The chelating agent-containing moiety is preferrably in aqueous solution at a pH of between about 5 and about 9, more preferably between pH about 6 to about 8. The chelating agent-containing moiety can be mixed with buffer salts such as citrate, acetate, phosphate and borate to produce the optimum pH. Preferably, the buffer salts are selected so as not to interfere with the subsequent binding of the metal ion to the chelating agent.

Where the imagable moiety contains a single chelant, that chelant may be attached directly to the nanoparticle of the present invention, eg. via one of the metal coordinating groups of the chelant which may form an ester, amide, thioester or thioamide bond with an amine, thiol or hydroxyl group on the nanoparticle. Alternatively the nanoparticle and chelant may be directly linked via a functionality attached to the chelant backbone, eg. a CH₂-phenyl-NCS group attached to a ring carbon of DOTA and DTPA as proposed by Meares et al. in JACS 110:6266-6267(1988), or indirectly via a homo or hetero-bifunctional linker, eg. a bis amine, bis epoxide, diol, diacid, difunctionalised PEG, etc.

### Non-metal Atomic Imagable Moiety:

Preferred non-metal atomic imagable moieties include radioisotopes such as ¹²³I and ¹³¹I as well as non zero nuclear spin atoms such as ¹⁸F, and heavy atoms such as I. Such imagable moieties, preferably a plurality thereof, eg. 2 to 200, may be covalently bonded to a linker backbone, either directly using conventional chemical synthesis techniques or via a supporting group, eq. a triiodophenyl group.

### Organic Chromophoric or Fluorophoric Imagable Moieties:

Preferred organic chromophoric and fluorophoric imagable moieties include groups having an extensive delocalized electron system, eg. cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes, etc. Examples of suitable organic or metallated organic chromophores may be found in “Topics in Applied Chemistry: Infrared absorbing dyes” Ed. M. Matsuoka, Plenum, NY 1990. Particular examples of chromophores which may be used have absorption maxima between 600 and 1000 nm to avoid interference with haemoglobin absorption. Further such examples include: cyanine dyes: such as heptamethinecyanine dyes. Specific dyes structures useful in the present invention are listed elsewhere in this specification.

### Administration to Human Body or Live Animals

The contrast agent of the present invention is preferably administered as a pharmaceutical formulation comprising the nanoparticle in a form suitable for administration to a mammal. The administration is suitable being carried out by injection or infusion of the formulation such as an aqueous solution. The formulation may contain one or more pharmaceutical acceptable additives and/or excipients e.g. buffers; solubilizers such as cyclodextrins; or surfactants such as Pluronic, Tween or phospholipids. Further, stabilizers or antioxidants such as ascorbic acid, gentisic acid or para-aminobenzoic acid and also bulking agents for lyophilisation such as sodium chloride or mannitol may be added.

The present invention also provides a pharmaceutical composition comprising an effective amount (e.g. an amount effective for enhancing image contrast in an in vivo imaging procedure) of a composition of the nanoparticle-based contrast agent of the present invention or a salt thereof, together with one or more pharmaceutically acceptable adjuvants, excipients or diluents.

A further aspect the invention provides the use of a composition of the nanoparticle-based contrast agent of the present invention for the manufacture of a contrast medium for use in a method of diagnosis involving administration of a contrast medium to a human or animal body and generation of an image of at least part of the body.

Still a further aspect of the invention provides a method of generating enhanced images of a human or animal body previously administered with the nanoparticle-based contrast agent composition which method comprises generating an image of at least part of the body.

### EXAMPLES

The following examples are provided to illustrate the invention.

### Example 1

### Synthesis of Compound 17

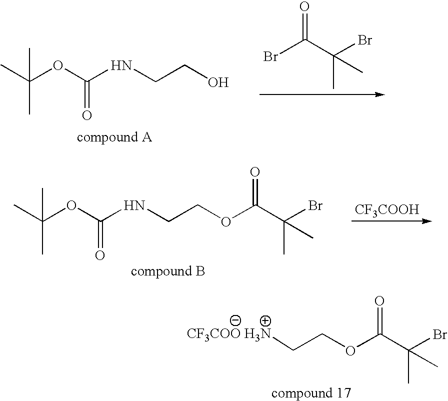

Compound B: Compound A (50 g, 0.31 mol) was dissolved in 300 mL of methylene chloride and triethylamine (34.5 g, 0.34 mol) was added to it. The solution was cooled in an ice-bath and 2-bromoisobutyryl bromide (71.3 g, 0.31 mol) in 150 mL of methylene chloride was added through an additional funnel. The reaction was slowly warmed up to room temperature and stirred at room temperature for 4 hours. The salt was filtered off and the reaction mixture was extracted with water and the organic phase was washed with saturated sodium bicarbonate solution and dried over magnesium sulfate. Solvent was evaporated and the crude product was purified by column chromatography using 80/20 heptane/diethyl ether as an eluent to give 65 g of pure product as a white solid, yield 92%. ¹H NMR (CDCl₃) δ (ppm): 1.45 (s, 9 H), 1.95 (s, 6 H), 3.42-3.48 (m, 2 H), 4.24 (t, J=5.25 Hz, 2 H).

Compound 17: Compound B (5.00 g, 0.016 mol) was added in portion to 15 mL of trifluoroacetic acid under vigorous stirring. The reaction was bubbling. After addition, the reaction was stirred at room temperature for 5 minutes and excess trifluoroacetic acid was removed. The crude product was dried under vacuum overnight, during which it slowly solidified. The crude product was stirred with a mixture of hexane and ethyl acetate and filtered to give 3.18 g of compound 17 as a white crystalline powder, yield 94%. ¹H NMR (CDCl₃) δ (ppm): 1.93 (s, 6 H), 3.35 (s, br, 2 H), 4.46 (t, J=5.1 Hz, 2 H), 8.10 (s, br, 3 H); ¹³C NMR (CDCl₃) δ (ppm): 30.24, 38.76, 55.52, 61.99, 161.49, 161.96, 162.43, 162.90, 171.78.

### Example 2

### Synthesis of Compound 18

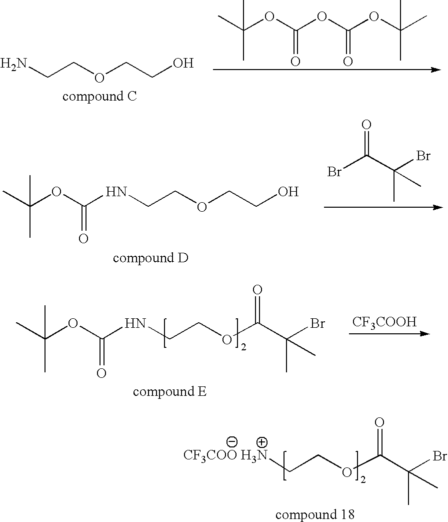

Compound D: compound C (10.0 g, 0.095 mol) was dissolved in 50 mL of methylene chloride and triethylamine (11.55 g, 0.114 mol) was added to it. The solution was cooled in an ice-bath and di-tert-butylcarbonate (22.83 g, 0.105 mol) in 30 mL of methylene chloride was added through an additional funnel. The reaction was slowly warmed up to room temperature and stirred at room temperature overnight. The reaction was extracted with water and the organic phase was washed with diluted HCl solution and dried over magnesium sulfate. Solvent was evaporated and the crude product was purified by column to give 12.43 g of pure product as an oil, 64% yield. ¹H NMR (CDCl₃) δ (ppm): 1.45 (s, 9 H), 3.30-3.35 (m, 2 H), 3.48-3.59 (m, 4 H), 3.71-3.76 (m, 2 H), 5.47 (s, br, 1 H).

Compound E: Compound D (12.43 g, 0.061 mol) was dissolved in 100 mL of methylene chloride and triethylamine (7.35 g, 0.073 mol) was added to it. The solution was cooled in an ice-bath and 2-bromoisobutyryl bromide (15.31 g, 0.067 mol) in 50 mL of methylene chloride was added through an additional funnel. The reaction was slowly warmed up to room temperature and stirred at room temperature overnight. The salt was filtered off and the reaction was extracted with water and the organic phase was washed with diluted HCl solution and dried over magnesium sulfate. Solvent was evaporated and the crude product was purified by column to give 13.5 g of pure product as an oil, 635 yield ¹H NMR (CDCl₃) δ (ppm): 1.44 (s, 9 H), 1.95 (s, 6 H), 3.29-3.34 (m, 2 H), 3.54-3.76 (m, 2 H), 3.69-3.72 (m, 2 H), 4.32-4.35 (m, 2 H), 5.0 (s, br, 1 H).

Compound 18: Compound E (13.0 g, 0.037 mol) was added in portion to 25 mL of trifluoroacetic acid under vigorous stirring. The reaction was bubbling. After addition, the reaction was stirred at room temperature for 5 minutes and excess trifluoroacetic acid was removed. The crude product was dried under vacuum overnight to give quantitative yield of product used without further purification. ¹H NMR (CDCl₃) δ (ppm): 1.91 (s, 6 H), 3.25-3.27 (m, 2 H), 3.74-3.77 (m, 4 H), 4.31-4.33 (m, 2 H), 7.40 (m, br, 3 H).

### Example 3

### Synthesis of Compound 1

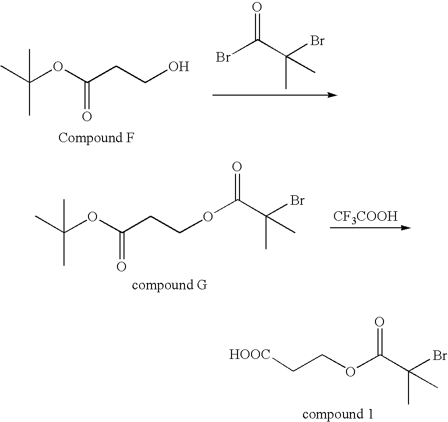

Compound G: Compound F (5.0 g, 0.034 mol) was dissolved in 50 mL of methylene chloride and triethylamine (4.15 g, 0.041 mol) was added to it. The solution was cooled in an ice-bath and 2-bromoisobutyryl bromide (8.65 g, 0.038 mol) in 10 mL of methylene chloride was added through an additional funnel. The reaction was slowly warmed up to room temperature and stirred at room temperature overnight. The reaction was extracted with water and the organic phase was washed with diluted HCl solution and dried over magnesium sulfate. Solvent was evaporated and the crude product was purified by column chromatography using 90/10 hexane/ethyl acetate as an eluent to give 8.70 g pure product as an oil, 86% yield. ¹H NMR (CDCl₃) δ (ppm): 1.47 (s, 9 H), 1.92 (s, 6 H), 2.62 (t, J=6.28 Hz, 2 H), 4.42 (t, J=6.28 Hz, 2 H).

Compound 1: Compound G (4.5 g, 0.015 mol) was dissolved in 20 mL of methylene chloride and trifluoroacetiec acid (31.3 g, 0.27 mol) added under vigorous stirring. The reaction was stirred at room temperature overnight and solvent and excess trifluoroacetic acid were removed. The crude product was dried under vacuum overnight, during which it slowly solidified. The crude product was stirred with a mixture of hexane and ethyl acetate and cooled down in a freezer. The white crystalline product was filtered to give 2.95 g of compound 1, 81% yield. ¹H NMR (CDCl₃) δ (ppm): 1.92 (s, 6 H), 2.78 (t, J=6.21 Hz, 2 H), 4.45 (t, J=6.21 Hz, 2 H).

### Example 4

### Synthesis of Compound 34

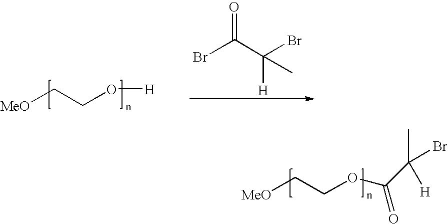

Poly(ethylene glycol) methyl ether (MW 5000, Aldrich, n=114) (50.0 g, 0.01 mol) was dried by dissolving in 200 mL of toluene. The solution was heated up with a Dean-Stark setup to remove water. After cooling down, toluene was removed and 200 mL of methylene chloride was added. The solution was cooled to 0° C. and triethylamine (3.0 g, 0.03 mol) was added. To the reaction was then added 2-bromopropionyl bromide (6.5 g, 0.03 mol). The reaction was stirred at room temperature overnight. The reaction was extracted with 1 N NaOH twice, 1 N HCl once, and brine once, and dried over MgSO₄. Solvent was reduced and the polymer was precipitated in diethyl ether twice to give off-white solid 44.0 g, 86% yield. ¹H NMR (CDCl₃) δ (ppm): 1.82 (d, J=6.88 Hz, 3 H), 1.42-1.47 (m, 1 H), 3.37 (s, 3 H), 3.65 (s, br, 454 H), 4.32-4.42 (m, 2 H).

Similarly, Poly(ethylene glycol) methyl ether PEG (MW 2000, Aldrich, n=45) was reacted with 2-bromopropionyl bromide to prepare the macroinitiator.

### Example 5

### Synthesis of Compound 36

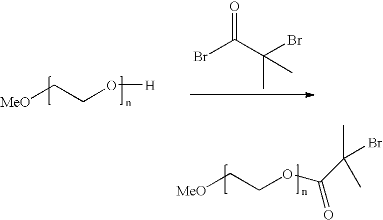

Poly(ethylene glycol) methyl ether PEG (MW 5000, Aldrich, n=114) (125.0 g, 0.025 mol) was dried by dissolving in 500 mL of toluene. The solution was heated up with a Dean-Stark setup to remove water. After cooling down, toluene was removed and 400 mL of methylene chloride was added. The solution was cooled to 0° C. and triethylamine (7.6 g, 0.075 mol) was added. To the reaction was then added 2-bromoisobutyryl bromide (17.2 g, 0.075 mol) in 20 mL of methylene chloride. The reaction was stirred at room temperature overnight. The reaction was extracted with 1 N NaOH twice, 1 N HCl once, and brine once, and dried over MgSO₄. Solvent was reduced and the polymer was precipitated in diethyl ether twice to give off-white solid 95.0 g, 74% yield. ¹H NMR (CDCl₃) δ (ppm): 1.94 (s, 6 H), 3.37 (s, 3 H), 3.65 (s, br, 454 H), 4.32-4.34 (m, 2 H).

Similarly, Poly(ethylene glycol) methyl ether PEG (MW 2000, Aldrich, n=45) was reacted with 2-bromoisobutyryl bromide to prepare the macroinitiator.

### Example 6

### Synthesis of Compound 40

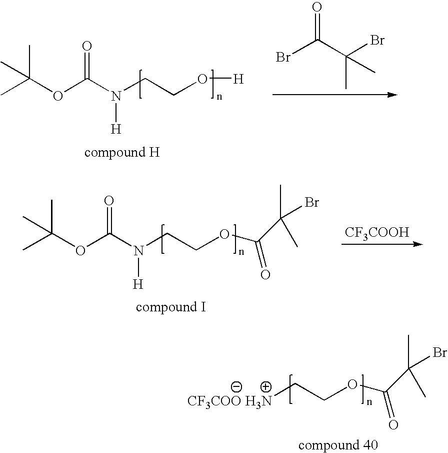

Compound I: Compound H (Intezyne Technologies LLC, Tampa, Fla.) (MW=5000, 10.0 g, 0.002 mol) was dissolved in 100 mL of methylene chloride and triethylamine (1.10 g, 0.011 mol) and catalytic amount (20 mg) of 4-N,N-dimethylaminopyridine were added to it. The solution was cooled in an ice-bath and 2-bromoisobutyryl bromide (2.4 g, 0.010 mol) in was added. The reaction was stirred at room temperature overnight. The reaction was extracted with 1 N NaOH twice, 1 N HCl once, and brine once, and dried over MgSO₄. Solvent was reduced and the polymer was precipitated in diethyl ether twice to give off-white solid 9.6 g. ¹H NMR (CDCl₃) δ (ppm): 1.44 (s, 9 H), 1.94 (s, 6 H), 3.65 (s, br, 454 H), 4.32-4.34 (m, 2 H).

Compound 40: Compound I (9.6 g, 0.002 mol) was dissolved in 50 mL of methylene chloride and trifluoroacetiec acid (2.5 g, 0.04 mol) added under vigorous stirring. The reaction was stirred at room temperature overnight and solvent and excess trifluoroacetic acid were removed. The residue was dissolved in methylene chloride and precipitated from diethyl ether twice to give 8.0 g of white solid. ¹H NMR (CDCl₃) δ (ppm): 1.94 (s, 6 H), 3.65 (s, br, 454 H), 4.32-4.34 (m, 2 H).

### Example 7

### Synthesis of Amine-terminated Poly(Ethylene Glycol) Macromonomer

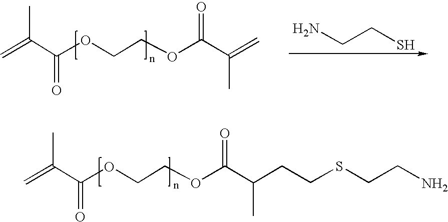

Polyethyleneglycol dimethacrylate (Aldrich, MW=875) 335 g was mixed with 100 ml of methanol and treated with cysteamine (Aldrich, MW 77) 5.8 g and diisopropylethylamine (Hunigs base) and was stirred at room temperature for 2 days and concentrated using a rotary evaporator. The residue was taken up in 1 L of ethyl acetate and extracted with aqueous 10% HCl. The aqueous layer was collected and made basic by the addition of 50% aqueous sodium hydroxide followed by extraction with ethyl acetate. The organic layer was dried over MgSO₄, filtered and concentrated. The residue was taken up in anhydrous diethyl ether and treated with gaseous HCl and allowed to stand. The ether was decanted to leave a dark blue oil. This material was washed with fresh diethyl ether, which was decanted. The dark blue oil was concentrated using a rotary evaporator to give 37 g of the desired product as the hydrochloride salt. ¹H NMR (300 MHz, CDCl₃) δ (ppm): 1.18 (d, 3 H), 1.93 (s, br, 3 H), 2.04 (s, br, 2 H), 2.43-2.77 (m, br, 7 H), 3.6-3.7 (s, b, —CH₂CH₂O—), 3.73 (t, br, 2 H), 3.29 (t, br, 2 H), 5.56 (s, br, 1 H), 6.12 (s, br, 1 H).

### Example 8

### Synthesis of Polymer 1-3

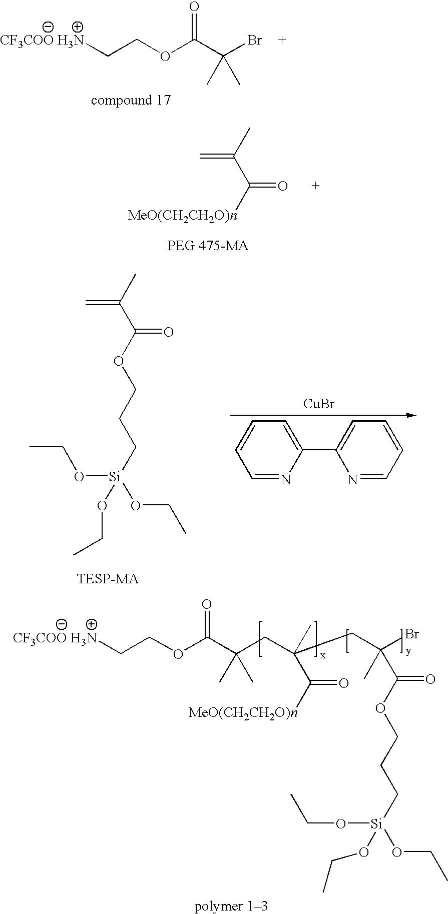

Polymer 1 (x=y=31): A 50 mL 3-neck flask was equipped with an additional funnel, a stopper, and a septum. Compound 17 (0.106 g, 0.33 mmol) and bipyridine (174 mg, 1.11 mmol) were dissolved in 2.4 mL of dry methanol in the flask, and methoxy-PEG 475 methacrylate (Aldrich, MW 475, passed through basic aluminum colum) (4.8 g, 0.01 mol) was added. Triethoxysilylpropyl methacrylate (PESP-MA) (Gelest, vacuum distilled from CaH₂) (3 mL, 2.94 g, 0.010 mol) was placed in the additional funnel with 1.5 mL of dry methanol. The additional funnel was equipped with a septum. Both mixtures in the flask and additional funnel were degassed by bubbling nitrogen for 15 minutes. Then CuBr (80 mg, 0.56 mmol) was added quickly to the flask and the reaction turned dark brown. The flask was heated in an oil bath at 50° C. for 30 minutes and PESP-MA solution was added quickly to the flask. The polymerization was continued overnight at 50° C. and was cooled down. The polymerization mixture was diluted with dry THF and it changed to bright green color. The mixture was passed through a pad of celite and neutral aluminum twice and concentrated to give clear viscous semi solid. ¹H NMR indicated the ratio of x/y to be close to 1/1. ¹H NMR (300 MHz, CDCl₃) δ (ppm): 0.55 (s, br), 0.77 (s, br), 0.95 (s, br), 1.18 (t, CH₃ from Si(OCH₂CH₃)), 1.64 (s, br), 1.96 (s, br), 3.31 (s, OMe), 3.58 (s, br, PEG), 3.75 (m, CH₂ from Si(OCH₂CH₃)), 4.01 (s, br).

Polymer 2 (x=31, y=16) and polymer 3 (x=31, y=46) were synthesized in a similar manner to polymer 1.

### Example 9

### Synthesis of Polymer 4 and 5

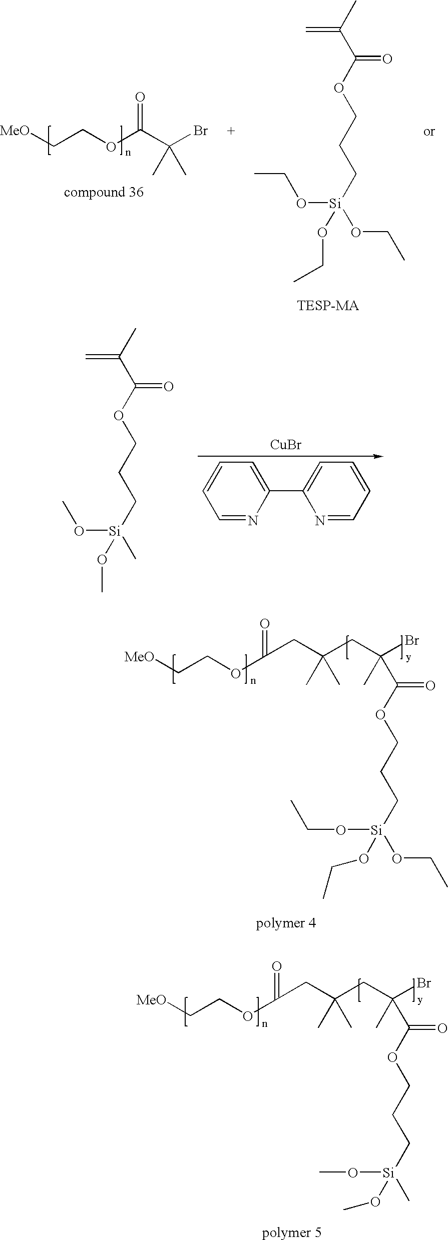

Polymer 4 (x=30, n=114): Compound 36 (1.10 g, 0.2 mmol) and bipyridine (76 mg, 0.49 mmol) were dissolved in 3 mL of dry methanol in a 25 mL round-bottomed the flask, and riethoxysilylpropyl methacrylate (PESP-MA) (2 mL, 1.96 g, 6.75 mmol) was added. The mixture was degassed by bubbling nitrogen for 15 minutes. Then CuBr (35 mg, 0.24 mmol) was added quickly to the flask and the reaction turned dark brown. The flask was heated in an oil bath at 50° C. for 5 hours during which the reaction became very viscous and magnetic stirred stopped. The polymerization was cooled down and diluted with dry THF. It changed to bright green color. The mixture was passed through a pad of celite and neutral aluminum twice and concentrated to give off-white semi-solid. ¹H NMR (300 MHz, CDCl₃) δ (ppm): 0.61 (s, br), 0.86 (s, br), 1.02 (s, br), 1.23 (t, CH₃ from Si(OCH₂CH₃)), 1.72 (s, br), 1.82 (s, br), 3.31 (s, OMe, very small), 3.65 (s, br, PEG), 3.81 (m, CH₂ from Si(OCH₂CH₃)).

Polymer 5 (x=50, n=114) were prepared in a similar manner to polymer 4. ¹H NMR (300 MHz, CDCl₃) δ (ppm): 0.14 (s, SiMe), 0.62 (s, br), 0.86 (s, br), 1.03 (s, br), 1.68 (s, br), 1.78 (s, br), 3.52 (s, SiOMe, 6 H), 3.65 (s, br, PEG), 3.89 (s, br).

### Example 10

### Synthesis of Polymer 6 and 7

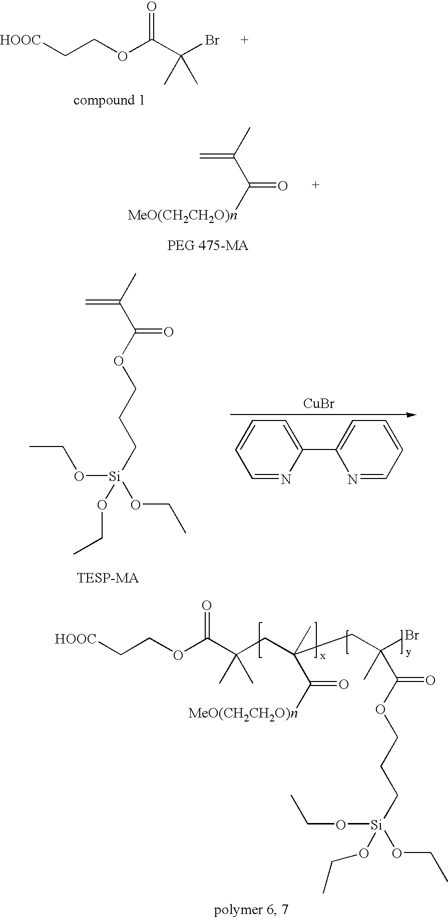

Polymer 6 (x=y=31) and polymer 7 (x=31, y=46) were synthesized in a similar manner to polymer 1.

### Example 11

### Synthesis of Silane Containing Near Infrared Dye 1

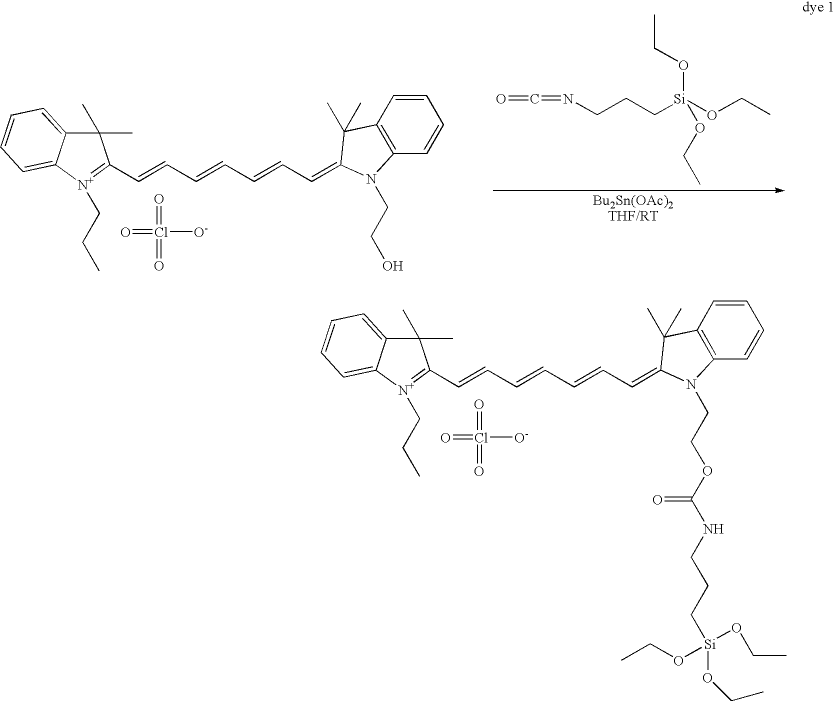

To a solution of dye precursor (1.13 g, 2 mmol) dissolved in THF (anhydrous, 20 ml) were added 3-(triethoxysilyl)propyl isocyanate (0.74 g, 3 mmol) and several drops of dibutyltin diacetate as a catalyst. The resulting mixture was stirred under N₂ at room temperature. The reaction was monitored by mass spectroscopy with completely disappearance of molecular weight of the dye precursor and appearance of the desired product. The solvent was removed by rotary evaporation. The residue was dissolved in small amount of dichloromethane and purified through silica gel chromatograph using a mixture of ethyl acetate and heptane as eluent to give pure product with extinction coefficient of 2.38×10⁵ Lmol⁻¹cm⁻¹.

### Example 12

### Synthesis of Near Infrared Dye Containing Nanoparticles

Polymer 3 (100 mg) and dye 1 (0.4 mg, 0.4 wt % to polymer 3) were dissolved in 10 mL of THF. 10 mL of distilled water was added slowly to the solution. The mixture was stirred at room temperature in the dark for 8 hours and acetic acid (0.05 mL) was added. The mixture was stirred at room temperature overnight. Most solvent was removed and the mixture was dialyzed using 3000 molecular weight cutoff tubing for 24 hours. The particle solution was filtered through 0.7, 0.45, 0.2 and 0.1 um filters. Particle size was measured using dynamic light scattering using Nanotrac from Microtrac, Inc. The volume average (MV) size of the particle is 31 nm.

The particles from the following polymers were made in a similar manner.

| polymer | Amount of dye to polymer (wt %) | MV (nm) |
|---|---|---|
| 1 | 0.25 | 27 |
| 1 | 0.50 | 20 |
| 2 | 0.25 | 22 |
| 2 | 0.50 | 15 |
| 3 | 0.25 | 35 |
| 3 | 0.50 | 33 |
| 3 | 0.15 | 34 |
| 3 | 0.35 | 34 |
| 3 | 0.05 | 31 |
| 3 | 0.10 | 32 |
| 3 | 0.40 | 31 |
| 3 | 0.20 | 33 |
| 3 | 0.35 | 31 |
| 4 | 3.00 | 24 |
| 5 | 0.50 | 32 |
| 5 | 1.00 | 22 |
| 5 | 2.00 | 19 |
| 5 | 3.00 | 17 |
| 5 | 5.00 | 19 |
| 6 | 0.40 | 19 |

### Example 13

### Comparative Example Showing Use of Sensitive Dyes

The following comparative example use the procedures generally outlined in U.S. Pat. No. 5,429,826 (see column 7, Method A). As previously discussed, when dye 1 is used in conjunction with the instant invention, good dye stability is observed. In contract, when dye 1 is used in conjunction with the prior art methods, poor dye stability is observed.

50 mg of polymer 1 and 0.2 mg of dye 1 (0.4 wt % to polymer) were dissolved in 10 mL of solvent (THF) and 0.25 mg of AIBN (2,2′-azobis(2-methyl propionitrile) was added. To the mixture was added 10 mL of water. The micelles were formed by removing solvent THF. The solution was deoxygenated and heated to 75° C. for 8 hours. The UV-Vis spectra of the solution was taken before and after the heating. Before heating the absorption at 753 nm was 1.092, after heating the deep blue-green dye color completely disappeared, and absorption at 746 nm was 0.046. Therefore, almost all the dye (95.8%) decomposed under such condition.

The solution was prepared the same as above. Instead of heating to 75° C., the solution was irradiated with UV light 254 nm for 2 hours. After irradiation, the deep blue-green dye color completely disappeared, and the absorption at 753 nm was 0.015. Therefore, almost all the dye (98.6%) decomposed under UV irradiation.

### Example 14

### Comparative Example Showing Use of Sensitive Dyes

The following comparative example use the procedures generally outlined in U.S. Pat. No. 5,429,826 (see column 7, Method A). As previously discussed, when dye 1 is used in conjunction with the instant invention, good dye stability is observed. In contract, when dye 1 is used in conjunction with the prior art methods, poor dye stability is observed.

The solution was prepared as in example 13 and the pH of the solution was adjusted to 5 by adding acetic acid. The solution stood at room temperature under ambient light for 5 days. The UV-Vis spectra of the solution was taken directly after the solution was prepared, after 1 day, and after 5 days. The absorption of the dye at 753 nm was 1.092, 1.031 (94.4%), and 0.461 (42.2%), respectively. Therefore, more than 50% of the dye decomposed after prolonged exposure to acidic condition.

### Example 15

### Synthesis of Diethylenetriamine Pentaacetic Acid (DTPA)-conjugated Nanoparticle

The nanoparticles from polymer 3 (0.4 wt % dye) synthesized in Example 12 (20 g, 9.3 mg/g nanoparticle concentration) were placed in an amber vial. The pH of the solution was adjusted to 7.5 using 2% sodium bicarbonate solution. Isothiocyanate-benzyl-DTPA (0.6 mg, prepared as 1 mg/ml in water, targeted to react 15% of amine end groups) was added to the vial. The reaction was stirred under nitrogen overnight. Free DTPA was removed by Centriprep® centrifugal filter unit with membrane having a 30,000 molecular weight cutoff and was analyzed by Capillary Electrophoresis. It was determined that 70% of DTPA was attached to the particles. Therefore, 10.5% of amine groups were attached with DTPA. The reaction was purified four times by centrifugation.

### Example 16

### Chelating of Gd to DTPA on Nanoparticles

To 5 mLs of 0.5 M NaOAc (the pH of this buffer is 8.5-8.6), was added 1 mL of the nanoparticles from example 15 (3% solids). To the mixture was added 2 eq. of Gd acetate to DTPA. The reaction was stirred at room temperature under nitrogen overnight. Free Gd acetate was removed by Centriprep® centrifugal filter unit with a membrane having a 30,000 molecular weight cutoff and repeated 4 times. The amount of chelated Gd was determined by ICP-AES (Inductively Coupled Plasma-Atomic Emission Spectrometer).

### Example 17

The nanoparticles from polymer 3 (0.4 wt % dye) synthesized in Example 12 (4.0 g, 9.3 mg/g nanoparticle concentration) were placed in an amber vial. The pH of the solution was adjusted to 7.5 using 2% sodium bicarbonate solution. Fluorescein-5 (6)-sothiocyanate (FITC) (0.091 mg, prepared as 0.5 mg/mL in THF, targeted to react 15% of amine end groups) was added to the vial. The reaction was stirred under nitrogen overnight. Free FITC was removed by Centriprep® centrifugal filter unit with membrane having a 30,000 molecular weight cutoff and was analyzed by UV-Vis. It was determined that 83% of DTPA was attached to the particles. Therefore, 12.5% of amine groups were attached with DTPA.

### Example 18

### Synthesis of Silane Containing NIR Dye II

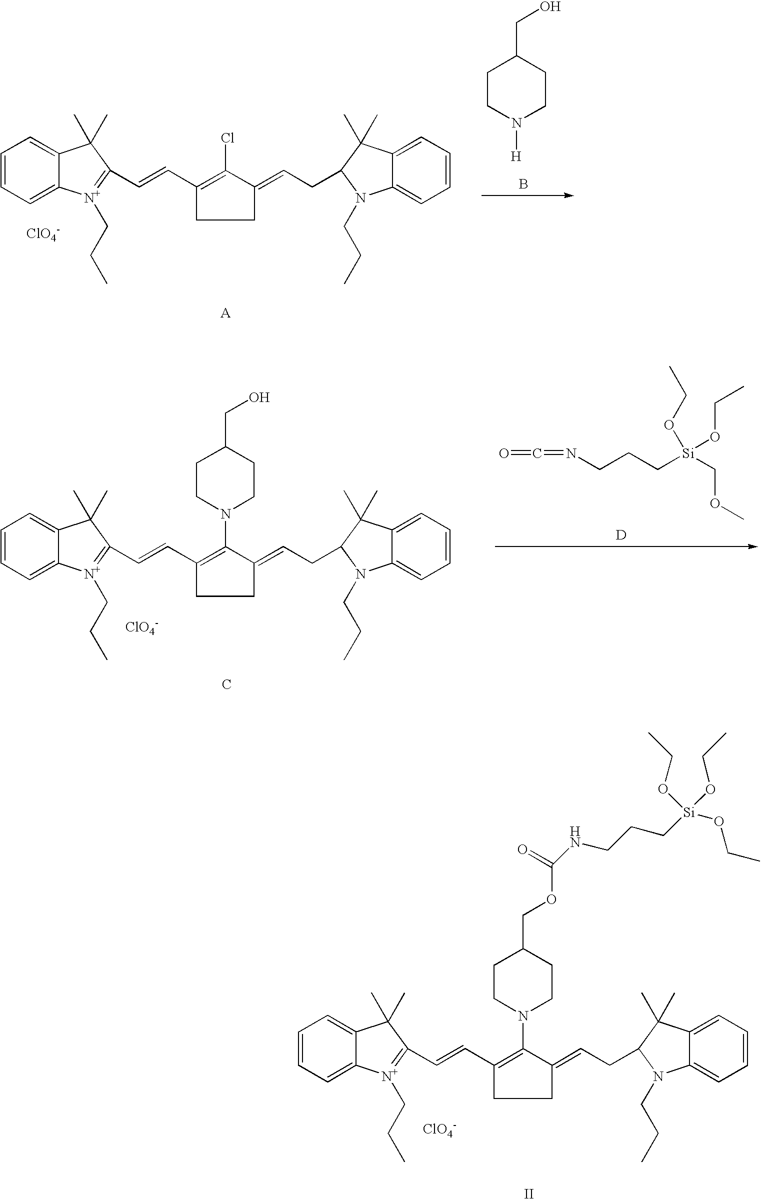

To a solution of dye A (3.2 g, 5.1 mmol) and B (0.9 g, 7.8 mmol) in DMSO (20 ml) was added K₂CO₃ (0.5 g). The resulting mixture was heated at 80° C. and monitored by UV-Vis with appearance of a new peak at 686 nm from dye C. Water was added to the mixture and crude dye C was precipitated out and collected by filtration. The dye was purified by silica gel chromatograph using a mixture of ethyl acetate and heptane, 1.2 g was obtained with correct mass.

To a solution of dye precursor C (1.2 g, 1.7 mmol) dissolved in THF (anhydrous, 40 ml) were added 3-(triethoxysilyl)propyl isocyanate D (2 ml, excess) and several drops of dibutyltin diacetate as a catalyst. The resulting mixture was stirred under N₂ at room temperature. The reaction was monitored by mass spectroscopy with completely disappearance of molecular weight of the dye precursor and appearance of the desired product. The solvent was removed by rotary evaporation. The residue was dissolved in small amount of dichloromethane and purified through silica gel chromatograph using a mixture of ethyl acetate and heptane as elutent to give pure product dye II (0.7 g) with absorption maximum of 688 nm; extinction coefficient of 1.19×10⁴ Lmol⁻¹cm⁻¹ and molecular weight of 953.7.

### Example 19

### Protein Binding Assay of Nanoparticles

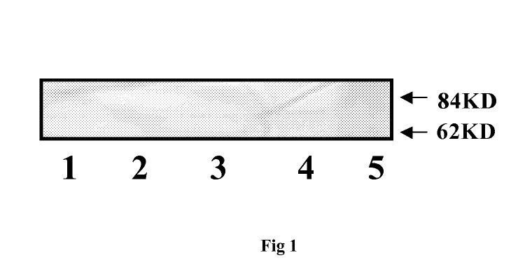

2 mg of each nanoparticle was mixed with 500 μl of BSA (bovine serum albumin) solution (40 mg/ml, physiological concentration), and was incubated for 1 hour at 37° C. with shaking at 850 rpm. After washing with phosphate-buffered saline buffer without calcium and magnesium (PBS (−)) for three times, sodium dodecyl sulfate (SDS)-sample buffer was added to the samples, then the samples were run on 10% SDS-polyacrylamide gel electrophoresis (SDS-PAGE) gel. The sample gel was stained with coomassie blue and scanned with Kodak imaging station 4000. Comparing with BSA (Control, 4 μg) as show in FIG. 1, nanoparticles of the present invention bind much less to serum protein, suggesting these particles could be quickly cleared (removed) from body.

In FIG. 1, illustrating protein binding assay of nanoparticles, run on 10% SDS-polyacrylamide gel electrophoresis (SDS-PAGE) gel, the following are represented:
1 particle 1=polymer 1, dye 0.5 wt %
2 particle 2=polymer 2, dye 0.5 wt %
3 particle 3=polymer 3, dye 0.5 wt %
4 control 4=4 μg
5 protein marker

### Example 20

### Cell Culture and Cytotoxicity Assay

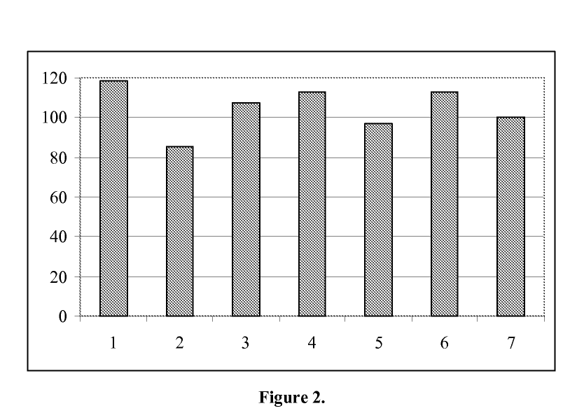

Human umbilical vein endothelial cells (HUVEC) were obtained from Cascade Biologics, Inc. (Portland, Oreg,) and maintained in Medium 200 containing 2% fetal bovine serum (FBS) with antibiotics, as described in manufacture instruction. The cells were studied at about 60-80% confluence between passages 3 and 7. To evaluate the cytotoxity of the nanoprticle, the CellTiter-Glo® Luminescent cell viability assay kit (Promega Corp., Madison, Wis.) was performed as described the manufacturer's instruction. This assay is a homogeneous method of determining the number of viable cells in culture based on quantification of the ATP present, an indicator of metabolically active cells. Nanoparticles were applied at different concentration 0.2 mg/ml and 0.02 mg/mL as seen in FIG. 2, nanoparticle 1, 2 and 3 did not show any significant cytotoxicity on the HUVEC.

In FIG. 2:
1 particle 1=polymer 1, dye 0.5 wt %, 0.2 mg/mL
2 particle 1=polymer 1, dye 0.5 wt %, 0.02 mg/mL
3 particle 2=polymer 2, dye 0.5 wt %, 0.2 mg/mL
4 particle 2=polymer 2, dye 0.5 wt %, 0.02 mg/mL
5 particle 3=polymer 3, dye 0.5 wt %, 0.2 mg/mL
6 particle 3=polymer 3, dye 0.5 wt %, 0.2 mg/mL
7 control=blank (no nanoparticles added)

The invention has been described in detail with particular reference to certain preferred embodiments thereof, but it will be understood that variations and modifications can be effected within the spirit and scope of the invention.

1. A nanoparticle comprising
a. a hydrophobic dye with a first silane moiety and
b. a copolymer which is comprised of
i. a hydrophilic block and
ii. a hydrophobic block with a second silane moiety cross-linked to the first silane moiety, wherein
c. the dye is immobilized in the hydrophobic block, and wherein
d. the copolymer self-assembles to form a nanoparticle when disposed in an aqueous medium such that the hydrophobic block defines a core region of the nanoparticle and the hydrophilic block defines an exterior region of the nanoparticle.

2. The nanoparticle as recited in claim 1, wherein the nanoparticle has a particle size of less than 100 nanometers.

3. The nanoparticle as recited in claim 2, wherein the copolymer is a block copolymer.

4. The nanoparticle as recited in claim 2, wherein the first and second silane moieties are each independently selected from the group consisting of an alkoxy silane moiety and an amino silane moiety.

5. The nanoparticle as recited in claim 2, wherein the first and second silane moieties are each alkoxy silane moieties.

6. The nanoparticle as recited in claim 1, wherein the first silane moiety is polyfunctional such that one first silane moiety is crosslinked to more than one second silane moiety.

7. The nanoparticle as recited in claim 1, wherein the second silane moiety is polyfunctional such that one second silane moiety is crosslinked to more than one first silane moiety.

8. The nanoparticle as recited in claim 1, wherein the hydrophilic block is comprised of an ethylene oxide moiety.

9. The nanoparticle as recited in claim 2, wherein the hydrophoblic block is comprised of a methacrylate moiety.

10. The nanoparticle as recited in claim 1, wherein the hydrophilic block is comprised of a reactive chemical unit on the exterior region of the nanoparticle.

11. The nanoparticle as recited in claim 10, wherein the reactive chemical unit is selected from the group consisting of a thiol, a carboxylic acid, an amine, and an activated ester.

12. A nanoparticle comprising
a. a hydrophobic dye with a first silane moiety and
b. a copolymer which is comprised of
i. a hydrophilic block and
ii. a hydrophobic block with a second silane moiety cross-linked to the first silane moiety, wherein
c. the first and second silane moieties are each independently selected from the group consisting of an alkoxy silane moiety and an amino silane moiety, and the first silane moiety is polyfunctional such that one first silane moiety is crosslinked to more than one second silane moiety,
d. the dye is immobilized in the hydrophobic block, and
e. the copolymer self-assembles to form a nanoparticle when disposed in an aqueous medium such that the hydrophobic block defines a core region of the nanoparticle and the hydrophilic block defines an exterior region of the nanoparticle.

13. The nanoparticle as recited in claim 12, further comprising an imagable moiety.

14. The nanoparticle as recited in claim 13, wherein the imagable moiety is selected from the group consisting of a chelated metal with an atomic number greater than 37; paramagnetic species; a superparamagnetic species, a ferromagnetic species; a ferromagnetic species; a radioactive isotope; a material which is a gas at 37° C., and a chromophore.

15. The nanoparticle as recited in claim 13, wherein the imagable moiety is selected from the group consisting of a chelated metal with an atomic number greater than 37; a paramagnetic metal; and a radioactive isotope.

16. A nanoparticle comprising
a. a hydrophobic dye with a first silane moiety and
b. a copolymer which is comprised of
i. a hydrophilic block with a reactive chemical unit selected from the group consisting of a thiol, a carboxylic acid, an amine, and an activated ester, and wherein the reactive chemical unit is tethered to a biologically active moiety,
ii. a hydrophobic block with a second silane moiety cross-linked to the first silane moiety, wherein
c. the first and second silane moieties are each independently selected from the group consisting of an alkoxy silane moiety and an amino silane moiety, and the first silane moiety is polyfunctional such that a single first silane moiety is crosslinked to more than one second silane moiety,
d. the dye is immobilized in the hydrophobic block,
e. the nanoparticle has a particle size of less than 100 nanometers, and
f. the copolymer self-assembles to form a nanoparticle when disposed in an aqueous medium such that the hydrophobic block defines a core region of the nanoparticle and the hydrophilic block defines an exterior region of the nanoparticle.

17. The nanoparticle as recited in claim 16, further comprising an imagable moiety selected from the group consisting of a chelated metal with an atomic number greater than 37; a paramagnetic metal; and a radioactive isotope.

18. The nanoparticle as recited in claim 16, wherein the biologically active moiety is a targeting molecule.

19. The nanoparticle as recited in claim 16, wherein the biologically active moiety is an antibody or antibody fragment.

20. The nanoparticle as recited in claim 16, wherein the biologically active moiety is a vitamin.
